(19) United States
(12) Patent Application Publication
(10) Pub. No.: US 2013/0209490 A1
(43) Pub. Date: Aug. 15, 2013

(54) Diagnostic BioMarkers for Fibrotic Disorders

(71) Applicants: Alan Buckler, Arlington, MA (US); Chao-Min Chen, Shanghai (CN); Chantale T. Guy, Medford, MA (US); Jeffrey Hewett, Duxbury, MA (US); Chris Lu, Shanghai (CN); Jing Wu, Shanghai (CN)

(72) Inventors: Alan Buckler, Arlington, MA (US); Chao-Min Chen, Shanghai (CN); Chantale T. Guy, Medford, MA (US); Jeffrey Hewett, Duxbury, MA (US); Chris Lu, Shanghai (CN); Jing Wu, Shanghai (CN)

(73) Assignee: NOVARTIS AG

(21) Appl. No.: 13/852,079

(22) Filed: Mar. 28, 2013

Related U.S. Application Data
(63) Continuation of application No. 13/319,133, filed on Nov. 7, 2011.

(51) Int. Cl. A61K 38/18; A61K 38/16; A61K 31/7088; A61K 45/06; A61K 39/395; G01N 33/50

(52) U.S. Cl. CPC A61K 38/1875; A61K 39/395; G01N 33/5091; A61K 31/7088; A61K 45/06; A61K 38/16
USPC 424/158.1; 435/6.1; 435/7.1; 435/7.9; 435/375; 514/8.8; 514/44 R

(57) ABSTRACT

The present invention provides novel methods of inhibiting fibrosis, as well as methods of treating or inhibiting fibrotic disorders, using BMP9 and/or BMP10 antagonists. The present invention also provides methods of assessing whether a subject has or is at risk of developing a fibrotic disorder by detecting levels of BMP9 and/or BMP10. Further provided are methods of assessing the efficacy of a treatment regimen for treating a fibrotic disorder by detecting and comparing pre-treatment levels of BMP9 and BMP10 with post-treatment levels of BMP9 and BMP10.

### BACKGROUND OF THE INVENTION

Fibrosis is a pathological process that refers to the aberrant formation or development of excess fibrous connective tissue by cells in an organ or tissue. Although processes related to fibrosis can occur as part of normal tissue formation or repair, dysregulation of these processes can lead to altered cellular composition and excess connective tissue deposition that progressively impairs tissue or organ function.

For example, more than five million people worldwide are afflicted with pulmonary fibrosis, which occurs when the air sacs of the lungs gradually become replaced by fibrotic tissue, causing an irreversible loss of the tissue's ability to transfer oxygen into the bloodstream. Additionally, hepatic fibrosis represents a major worldwide healthcare burden and results from excessive connective tissue formation in the liver which can lead to portal hypertension or even cirrhosis (see Murphy et al., Expert Opin Investig Drugs. 2002 November; 11(11): 1575-85). Additionally, many other fibrotic disorders exist, including vascular fibrosis, pancreatic fibrosis, renal fibrosis, musculoskeletal fibrosis, cardiac fibrosis, skin fibrosis, eye fibrosis, progressive systemic sclerosis (PSS), chronic graft versus-host disease, Peyronie's disease, post-cystoscopic urethral stenosis, idiopathic and pharmacologically induced retroperitoneal fibrosis, mediastinal fibrosis, progressive massive fibrosis, proliferative fibrosis and neoplastic fibrosis.

Although therapeutic agents, such as anti-inflammatory drugs, are often used to treat fibrosis, such treatments can have low efficacy and undesirable side effects. Moreover, there are currently no wholly effective treatments or cures for fibrotic disorders. Accordingly, there is a great need in the art for moieties which can inhibit fibrosis and, therefore, can be used to treat or prevent fibrosis in a subject, as well as methods for diagnosing this debilitating disease.

### SUMMARY OF THE INVENTION

The present invention provides methods for inhibiting the fibrotic activity of a cell, methods for treating or preventing fibrosis in a subject and methods for assessing whether a subject has or is at risk for developing a fibrotic disorder. The present invention is based, at least in part, on the demonstration by the present inventors that BMP9 and BMP10 play a previously unknown role in the differentiation of fibroblasts and the excessive synthesis and accumulation of extracellular matrix and tissue remodeling by myofibroblasts during fibrosis.

Accordingly, the present invention provides novel methods of inhibiting fibrosis in a cell by contacting the cell with a BMP9 or BMP10 antagonist. In a particular embodiment, the cell includes, but is not limited to, a pulmonary cell, a vascular cell, a brain cell, a bone cell, a stem cell, a liver cell, a kidney cell, a cardiac cell, a musculoskeletal cell, a skin cell, an eye cell, or a pancreatic cell.

In another aspect, the present invention provides methods for treating or preventing a fibrotic disorder in a subject. The methods include administering to a subject, e.g., a human or an animal, an effective amount of a bone morphogenetic protein 9 (BMP9) or bone morphogenetic protein 10 (BMP10) antagonist. In one embodiment, the fibrotic disorder includes, but is not limited to, vascular fibrosis, pulmonary fibrosis (e.g., idiopathic pulmonary fibrosis), pancreatic fibrosis, liver fibrosis (e.g., cirrhosis), renal fibrosis, musculoskelctal fibrosis, cardiac fibrosis (e.g., endomyocardial fibrosis, idiopathic myocardiopathy), skin fibrosis (e.g., scleroderma, post-traumatic, operative cutaneous scarring, keloids and cutaneous keloid formation), eye fibrosis (e.g., glaucoma, sclerosis of the eyes, conjunctival and comeal scarring, and pterygium), progressive systemic sclerosis (PSS), chronic graft versus-host disease, Peyronie's disease, post-cystoscopic urethral stenosis, idiopathic and pharmacologically induced retroperitoneal fibrosis, mediastinal fibrosis, progressive massive fibrosis, proliferative fibrosis, neoplastic fibrosis, Dupuytren's disease, strictures, and radiation induced fibrosis. In a particular embodiment, the fibrotic disorder is not myelofibrosis.

In a particular embodiment, the present invention provides a method of treating or preventing a fibrotic disorder in a subject, comprising administering to the subject an effective amount of an anti-BMP9 or anti-BMP10 antibody, wherein the fibrotic disorder is selected from the group consisting of liver fibrosis, kidney fibrosis, heart fibrosis, skin fibrosis, and lung fibrosis.

In another embodiment, the present invention provides a method of inhibiting or preventing epithelial-mesenchymal transition (EMT) in a subject, comprising administering to the subject an effective amount of BMP9 or BMP10 antagonist, thereby inhibiting or preventing epithelial-mesenchymal transition (EMT).

In a further aspect, the present invention provides methods of assessing whether a subject has or is at risk of developing a fibrotic disorder. The methods include contacting a sample from a subject with a reagent able to detect BMP9 or BMP10 and detecting BMP9 or BMP10, wherein an elevated level of BMP9 or BMP10 activity (e.g., at least 1.5, 2, 2.5, 3, 3.5, 4, 4.5, 5-fold higher relative to the control) is an indication that the subject has or is at risk of developing a fibrotic disorder. In a particular embodiment, the method includes a further step of detecting the presence of additional fibrosis markers, e.g., alpha smooth muscle actin, collagen type III cartilage oligomeric matrix protein, collagen type, collagen type IV, fibroblast specific protein-1, fibronectin, serpinE1, periostin, IGFBP3, SPARC, CTGF, TGFb, Cyr61, and phospho smad 2/3.f

In one embodiment, the sample includes cells or tissues obtained from the subject. In another embodiment, the sample is a fluid (e.g., blood fluid, lymph, gynecological fluid, cystic fluid, ocular fluid, urine, and fluid collected by peritoneal rinsing) obtained from the subject.

The reagents encompassed by the methods of the invention include any known agent capable of detecting BMP9 and/or BMP10. In one embodiment, the reagent is an antibody. In another, the reagent is a nucleic acid. The reagents may also be labeled (e.g., a radioisotope, a bioluminescent compound, a chemiluminescent compound, a fluorescent compound, a metal chelate, or an enzyme), so as to facilitate detection of BMP9 and/or BMP10.

In yet a further aspect, the present invention provides methods of assessing the efficacy of a treatment regimen for treating a fibrotic disorder in a subject. These methods include contacting a first sample obtained from the subject prior to administering at least a portion of the treatment regimen to the subject with a reagent able to detect BMP9 or BMP10, contacting a second sample obtained from the subject following administration of at least a portion of the treatment regimen with a reagent able to detect BMP9 or BMP10, comparing the levels of BMP9 or BMP10 activity from the first and second samples, wherein an elevated level of BMP9 or BMP10 present in the first sample, relative to the second sample, is an indication that the treatment regimen is efficacious for treating a fibrotic disorder in the subject. In a particular embodiment, the treatment regimen comprises administration of a BMP9 or BMP10 antagonist.

In another aspect, the present invention provides methods for inhibiting the differentiation of a fibroblast or other cell to a myofibroblast, by contacting a fibroblast with an effective amount of a BMP9 or BMP10 antagonist.

In one aspect, the invention is directed to the use of a BMP9 or BMP10 antagonist in inhibiting the fibrotic response of a cell, e.g., a pulmonary cell, a liver cell, a kidney cell, a cardiac cell, a musculoskeletal cell, a skin cell, an eye cell, and a pancreatic cell.

In another aspect, the invention is directed to the use of a BMP9 or BMP10 antagonist in treating a fibrotic disorder in a subject.

In another embodiment, the invention is directed to the use of a BMP9 or BMP10 antagonist in preventing a fibrotic disorder in a subject.

In a further embodiment, the invention is directed to the use of a BMP9 or BMP10 antagonist in inhibiting the differentiation of a fibroblast or other cell to a myofibroblast.

The invention is also directed to the use of a reagent for assessing whether a subject has or is at risk of developing a fibrotic disorder, the use comprising: (i) contacting a sample from said subject with a reagent able to detect BMP9 or BMP10 or their biological activity; and (ii) detecting BMP9 or BMP10, wherein an elevated level of BMP9 or BMP10 activity relative to a control is an indication that the subject has or is at risk of developing a fibrotic disorder.

In a further embodiment, the invention is directed to the use of a reagent for assessing the efficacy of a treatment regimen for treating a fibrotic disorder in a subject, the use comprising: a) contacting a first sample obtained from said subject prior to administering at least a portion of the treatment regimen to the subject with a reagent able to detect BMP9 or BMP10; b) contacting a second sample obtained from said subject following administration of at least a portion of the treatment regimen with a reagent able to detect BMP9 or BMP10; and c) comparing the levels of BMP9 or BMP10 from the first and second samples, wherein an elevated level of BMP9 or BMP10 present in the first sample, relative to the second sample, is an indication that the treatment regimen is efficacious for treating a fibrotic disorder in the subject.

A wide variety of BMP9 and BMP10 antagonists can be used in the methods of the present invention, such as antibodies, fusion proteins, adnectins, aptamers, anticalins, lipocalins, nucleic acids (e.g., antisense molecules, such as RNA interfering agents and ribozymes), immunoconjugates (e.g., an antibody conjugated to a therapeutic agent), small molecules, fusion proteins, BMP9 or BMP10-derived peptidic compounds, and receptor-based antagonists (e.g., soluble ALK1 or serine/threonine kinase receptors).

In a particular embodiment, the BMP9 or BMP10 antagonist is an antibody, or fragment thereof. Antibodies suitable for protection according to the invention include all known forms of antibodies having at least variable region sequences. For example, the antibody can be a murine, human, humanized, chimeric or bispecific monoclonal antibody. The antibody can be a Fab, Fab′2, ScFv, SMIP, affibody, avimer, versabody, nanobody, and a domain antibody and the antibody can be an IgG1, IgG2, IgG3, IgG4, IgM, IgA1, IgA2, IgAsec, IgD, or IgE antibody.

BMP9 or BMP10 antagonists used in the methods of the present invention can be administered via any suitable method including, but not limited to intravenous, intramuscular or subcutaneous administration.

Other features and advantages of the invention will be apparent from the following detailed description, and from the claims.

### BRIEF DESCRIPTION OF THE DRAWINGS

FIG. 1 depicts the results of a high content immunostaining experiment leading to the identification of BMP9 (i.e., GDF2) and BMP10 as therapeutic targets capable of treating fibrotic disorders.

FIG. 2 is a graph depicting BMP9 and BMP10 activity on myofibroblastic differentiation of fibroblasts in a High Throughput Screening (HTS) assay.

FIG. 3 is a graph depicting the upregulation of collagen type III, a known marker of fibrosis, after treatment of fibroblasts with recombinant BMP9 or BMP10.

FIG. 4 is a graph depicting the upregulation of alpha smooth actin, a known marker of fibrosis, after treatment of fibroblasts with recombinant BMP9 or BMP10.

FIG. 5 is a graph depicting upregulation of cartilage oligomeric matrix protein (COMP), a known marker of fibrosis, after treatment of fibroblasts with recombinant BMP9 or BMP10.

FIG. 6 is a graph depicting the effects of an anti-BMP9 monoclonal antibody and an ALK1-Fc soluble receptor on BMP9-stimulated BRE-luciferase activity in a reporter gene assay.

FIG. 7 depicts the morphological and immunohistochemical changes in Hep3B cells treated with or without BMP9 in an Epithelial-Mesenchymal Transdifferentiation (EMT) assay.

FIGS. 8A-8D are graphs depicting the effect of BMP9 on the expression of four different fibroblast markers (i.e., αSMA, Col la1, fibroblast specific protein, and vimentin) in an EMT assay.

FIG. 9 depicts the effects of a BMP9 neutralizing monoclonal antibody and a soluble receptor (ALK1-Fc) on BMP9-induced FSP-1 expression in an EMT assay.

### DETAILED DESCRIPTION OF THE INVENTION

The present invention provides methods for inhibiting the fibrotic response of a cell, methods for treating or preventing a fibrotic disorder in a subject and methods for assessing whether a subject has or is at risk for developing a fibrotic disorder. The present invention is based, at least in part, on the demonstration by the present inventors that BMP9 and BMP10 play a previously unknown role in the differentiation of fibroblasts into myofibroblasts during the development of fibrosis.

In order that the present invention may be more readily understood, certain terms are first defined. Additional definitions are provided throughout the detailed description.

### I. DEFINITIONS

As used herein, the term bone morphogenetic proteins (also referenced interchangeably herein as “BMPs”) refers to a group of multi functional growth factors and cytokines known for their ability to induce the formation of bone and cartilage. To date, approximately 20 BMPs have been identified. With the exception of bone morphogenetic protein 1 (BMP1), all of the bone morphogenetic proteins belong to the transforming growth factor beta (TGF β) superfamily (see Chen et al., Growth Factors. 2004 December; 22(4):233-41).

As used herein, the term bone morphogenetic protein 9 (also referenced interchangeably herein as “BMP9”, “BMP-9”, “growth differentiation factor 2”, “GDF-2”, “GDF2,” and “Growth/differentiatiation factor 2 precursor” refers to the art known member of the TGFβ/BMP superfamily that is known to be a potent inducer of osteoblast differentiation of mesenchymal stem cells (see Tang et al. (2008) J Cell Mol Med. [PMID: 19175684]). BMP9 has also been shown to be involved in the regulation of glucose metabolism, capable of reducing glycemia in diabetic mice, a differentiation factor for cholinergic neurons in the central nervous system, and to induce the expression of a hormone (hepcidin) that plays a role in iron homeostatis (David et al. 2008. Circ Res. April 25; 102(8):914-22).

A representative BMP9 sequence, includes, but is not limited to, the sequence set forth below.

BMP9/Growth Differentiation Factor 2 [Homo sapiens] (NP_057288)
(SEQ ID NO: 1)
MCPGALWVALPLLSLLAGSLQGKPLQSWGRGSAGGNAHSPLGVPGGGLPE
HTFNLKMFLENVKVDFLRSLNLSGVPSQDKTRVEPPQYMIDLYNRYTSDKSTTPA
SNIVRSFSMEDAISITATEDFPFQKHILLFNISIPRHEQITRAELRLYVSCQNHVDPSH
DLKGSVVIYDVLDGTDAWDSATETKTFLVSQDIQDEGWETLEVSSAVKRWVRSD
STKSKNKLEVTVESHRKGCDTLDISVPPGSRNLPFFVVFSNDHSSGTKETRLELRE
MISHEQESVLKKLSKDGSTEAGESSHEEDTDGHVAAGSTLARRKRSAGAGSHCQ
KTSLRVNFEDIGWDSWIIAPKEYEAYECKGGCFFPLADDVTPTKHAIVQTLVHLK
FPTKVGKACCVPTKLSPISVLYKDDMGVPTLKYHYEGMSVAECGCR
BMP9/Growth Differentiation Factor 2 [Homo sapiens] (AF188285)
(SEQ ID NO: 2)
cggtccagcc cggcagcggg tgagagtagg tgctggccaa gacggttcct tcagagcaaa
cagcagggag atgccggccc gctccttccc agctcctccc cgtgcccgct aacacagcac
ggccgcctgc agtctcctct ctgggtgatt gcgcgggcct aagatgtgtc ctggggcact
gtgggtggcc ctgcccctgc tgtccctgct ggctggctcc ctacagggga agccactgca
gagctgggga cgagggtctg ctgggggaaa cgcccacagc ccactggggg tgcctggagg
tgggctgcct gagcacacct tcaacctgaa gatgtttctg gagaacgtga aggtggattt
cctgcgcagc cttaacctga gtggggtccc ttcgcaggac aaaaccaggg tggagccgcc
gcagtacatg attgacctgt acaacaggta cacgtccgat aagtcgacta cgccagcgtc
caacattgtg cggagcttca gcatggaaga tgccatctcc ataactgcca cagaggactt
ccccttccag aagcacatct tgctcttcaa catctccatt cctaggcatg agcagatcac
cagagctgag ctccgactct atgtctcctg tcaaaatcac gtggacccct ctcatgacct
gaaaggaagc gtggtcattt atgatgttct ggatggaaca gatgcctggg atagtgctac
agagaccaaa accttcctgg tgtcccagga cattcaggat gagggctggg agaccttgga
agtgtccagc gccgtgaagc gctgggtccg gtccgactcc accaagagca aaaataagct
ggaagtgact gtggagagcc acaggaaggg ctgcgacacg ctggacatca gtgtcccccc
aggttccaga aacctgccct tctttgttgt cttctccaat gaccacagca gtggaaccaa
ggagaccagg ctggagctga gggagatgat cagccatgaa caagagagcg tgctcaagaa
gctgtccaag gacggctcca cagaggcagg tgagagcagt cacgaggagg acacggatgg
ccacgtggct gcggggtcga ctttagccag gcggaaaagg agcgccgggg ctggcagcca
ctgtcaaaag acctccctgc gggtaaactt cgaggacatc ggctgggaca gctggatcat
tgcacccaag gagtatgaag cctacgagtg taagggcggc tgcttcttcc ccttggctga
cgatgtgacg ccgacgaaac acgctatcgt gcagaccctg gtgcatctca agttccccac
aaaggtgggc aaggcctgct gtgtgcccac caaactgagc cccatctccg tcctctacaa
ggatgacatg gaggtgccca ccctcaagta ccattacgag ggcatgagcg tggcagagtg
tgggtgcagg tagtatctgc ctgcggggct ggggaggcag gccaaagggg ctccacatga
gaggtcctgc atgcccctgg gcacaacaag gactgattca atctgcatgc cagcctggag
gaggaaaggg agcctgctct ccctccccac accccaccca aagcatacac cgctgagctc
aactgccagg gaaggctaag gaaatgggga tttgagcaca acaggaaagc ctgggagggt
tgttgggatg caaggaggtg atgaaaagga gacaggggga aaaataatcc atagtcagca
gaaaacaaca gcagtgagcc agaggagcac aggcgggcag gtcactgcag agactgatgg
aagttagaga ggtggaggag gccagctcac tccaaaaccc ttggggagta gagggaagga
gcaggccgcg tgtcacaccc atcattgtat gttatttccc acaacccagt tggaggggca
tggcttccaa tttagagacc cg

The murine and other animal BMP9 molecules are known in the art (see, for example, NP_{—}062379 for murine BMP9 and NP_{—}001099566 for rat BMP9).

As used herein, the term bone morphogenetic protein 10 (also referenced interchangeably herein as “BMP10”, “BMP-10”. “MGC126783”, and “Bone morphogenetic protein 10 precursor” refers to art known member of the TGFβ/BMP superfamily. It has been suggested that BMP10 is an essential component in modulating cardiomyocyte proliferation and maturation during cardiac ventricular development. (Chen et al., (2004) Development. 131(9):2219-31 and Neubaus et al., (1999) Mech Dev., 80(2): 181-4).

A representative BMP10 sequence, includes, but is not limited to, the sequence set forth below.

Bone Morphogenetic Protein 10 Preproprotein [Homo sapiens] (NP_055297)
(SEQ ID NO: 3)
MGSLVLTLCALFCLAAYLVSGSPIMNLEQSPLEEDMSLFGDVFSEQDGVDFNTLL
QSMKDEFLKTLNLSDIPTQDSAKVDPPEYMLELYNKFATDRTSMPSANIIRSFKNE
DLFSQPVSFNVSIPHHEEVIMAELRLYTLVQRDRMIYDGVDRKITTFEVLESKGDN
EGERNMLVLVSGEIYGTNSEWETFDVTDAIRRWQKSGSSTHQLEVHIESKHDEAE
DASSGRLEIDTSAQNKHNPLLIVFSDDQSSDKERKEELNEMISHEQLPELDNLGLD
SFSSGPGEEALLQMRSNIIYDSTARIRRNAKGNYCKRTPLYIDFKEIGWDSWIIAPP
GYEAYECRGVCNYPLAEHLTPTKHAIIQALVHLKNSQKASKACCVPTKLEPISILY
LDKGVVTYKFKYEGMAVSECGCR
Bone Morphogenitic Protein 10 [Homo sapiens] (NM_014482)
(SEQ ID NO: 4)
ggggagagga agagtggtag ggggagggag agagagagga agagtttcca aacttgtctc
cagtgacagg agacatttac gttccacaag ataaaactgc cacttagagc ccagggaagc
taaaccttcc tggcttggcc taggagctcg agcggagtca tgggctctct ggtcctgaca
ctgtgcgctc ttttctgcct ggcagcttac ttggtttctg gcagccccat catgaaccta
gagcagtctc ctctggaaga agatatgtcc ctctttggtg atgttttctc agagcaagac
ggtgtcgact ttaacacact gctccagagc atgaaggatg agtttcttaa gacactaaac
ctctctgaca tccccacgca ggattcagcc aaggtggacc caccagagta catgttggaa
ctctacaaca aatttgcaac agatcggacc tccatgccct ctgccaacat cattaggagt
ttcaagaatg aagatctgtt ttcccagccg gtcagtttta atgggctccg aaaatacccc
ctcctcttca atgtgtccat tcctcaccat gaagaggtca tcatggctga acttaggcta
tacacactgg tgcaaaggga tcgtatgata tacgatggag tagaccggaa aattaccatt
tttgaagtgc tggagagcaa aggggataac gagggagaaa gaaacatgct ggtcctggtg
tctggggaga tatatggaac caacagtgag tgggagactt ttgatgtcac agatgccatc
agacgttggc aaaagtcagg ctcatccacc caccagctgg aggcccacat tgagagcaaa
cacgatgaag ctgaggatgc cagcagtgga cggctagaaa tagataccag tgcccagaat
aagcataacc ctttgctcat cgtgttttct gatgaccaaa gcagtgacaa ggagaggaag
gaggaactga atgaaatgat ttcccatgag caacctccag agctggacaa cttgggcctg
gatagctttt ccagtggacc tggggaagag gctttgttgc agatgagatc aaacatcatc
tatgactcca ctgcccgaat cagaaggaac gccaaaggaa actactgtaa gaggaccccg
ctctacatcg acttcaagga gattgggtgg gactcctgga tcatcgctcc gcctggatac
gaagcctatg aatgccgtgg tgtttgtaac taccccctgg cagagcatct cacacccaca
aagcatgcaa ttatccaggc cttggtccac ctcaagaatt cccagaaagc ttccaaagcc
tgctgtgtgc ccacaaagct agagcccatc tccatcctct atttagacaa aggcgtcgtc
acctacaagt ttaaatacga aggcatggcc gtctccgaat gtggctgtag atagaagaag
agtcctatgg cttatttaat aactgtaaat gtgtatattt ggtgttccta tttaatgaga
ttatttaata agggtgtaca gtaatagagg cttgctgcct tcaggaaatg gacaggtcag
tttgttgtag gaaatgcata tttt

The murine and other animal BMP10 molecules are known in the art (see, for example, NP_{—}033886 for murine BMP10).

As used herein, the term “antagonist” refers to any moiety which downregulates BMP9 and/or BMP10 activity, including agents which downregulate BMP9 and/or BMP10 expression or inhibit BMP9 and/or BMP10 function.

As used herein, the term “downregulates” refers to any statistically significant decrease in a biological activity and/or expression of BMP9 and/or BMP10, including full blocking of the activity (i.e., complete inhibition) and/or expression. For example, “downregulation” can refer to a decrease of about 10%, 20%, 30%, 40%, 50%, 60%, 70%, 80%, 90%, or 100% in BMP9 and/or BMP10 activity and/or expression.

As used herein, the term “inhibit” or “inhibiting” fibrosis refers to any statistically significant decrease in a biological activity and/or expression of BMP9 and/or BMP10, including full blocking of the activity and/or expression. For example, “inhibition” can refer to a decrease of about 10%, 20%, 30%, 40%, 50%, 60%, 70%, 80%, 90%, or 100% in BMP9 and/or BMP10 activity and/or expression.

As used herein, the term “fibrosis” refers to the aberrant formation or development of excess fibrous connective tissue by cells in an organ or tissue. Although processes related to fibrosis can occur as part of normal tissue formation or repair, dysregulation of these processes can lead to altered cellular composition and excess connective tissue deposition that progressively impairs to tissue or organ function. There are several types of fibrosis, for example, cystic fibrosis of the pancreas and lungs, injection fibrosis, which can occur as a complication of intramuscular injections, especially in children, endomyocardial fibrosis, idiopathic pulmonary fibrosis of the lung, mediastinal fibrosis, myelofibrosis, retroperitoneal fibrosis, progressive massive fibrosis, a complication of coal workers' pneumoconiosis, and nephrogenic systemic fibrosis.

As used herein, the terms “fibrotic disorder”, “fibrotic condition,” and “fibrotic disease,” are used interchangeably to refer to a disorder, condition or disease characterized by fibrosis. Examples of fibrotic disorders include, but are not limited to vascular fibrosis, pulmonary fibrosis (e.g., idiopathic pulmonary fibrosis), pancreatic fibrosis, liver fibrosis (e.g., cirrhosis), renal fibrosis, musculoskeletal fibrosis, cardiac fibrosis (e.g., endomyocardial fibrosis, idiopathic myocardiopathy), skin fibrosis (e.g., scleroderma, post-traumatic, operative cutaneous scarring, keloids and cutaneous keloid formation), eye fibrosis (e.g., glaucoma, sclerosis of the eyes, conjunctival and corneal scarring, and pterygium), progressive systemic sclerosis (PSS), chronic graft versus-host disease, Peyronie's disease, post-cystoscopic urethral stenosis, idiopathic and pharmacologically induced retroperitoneal fibrosis, mediastinal fibrosis, progressive massive fibrosis, proliferative fibrosis and neoplastic fibrosis.

As used herein, the term “cell” refers to any cell prone to undergoing a fibrotic response, including, but not limited to, individual cells, tissues, and cells within tissues and organs. The term cell, as used herein, includes the cell itself, as well as the extracellular matrix (ECM) surrounding a cell. For example, inhibition of the fibrotic response of a cell, includes, but is not limited to the inhibition of the fibrotic response of one or more cells within the lung (or lung tissue); one or more cells within the liver (or liver tissue); one or more cells within the kidney (or renal tissue); one or more cells within muscle tissue; one or more cells within the heart (or cardiac tissue); one or more cells within the pancreas; one or more cells within the skin; one or more cells within the bone, one or more cells within the vascalature, one or more stem cells, or one or more cells within the eye.

As used herein, the term “Epithelial-Mesenchymal Transition” (EMT) refers to the conversion from an epithelial to a mesenchymal phenotype, which is a normal process of embryonic development. EMT is also the process whereby injured epithelial cells that function as ion and fluid transporters become matrix remodeling mesenchymal cells. In carcinomas, this transformation results in altered cell morphology, the expression of mesenchymal proteins and increased invasiveness. The criteria for defining EMT in vitro involve the loss of epithelial cell polarity, the separation into individual cells and subsequent dispersion after the acquisition of cell motility (See Vincent-Salomon et al., Breast Cancer Res. 2003; 5(2): 101-106). Classes of molecules that change in expression, distribution, and/or function during EMT, and that are causally involved, include growth factors (e.g., transforming growth factor (TGF)-β, wnts), transcription factors (e.g., snails, SMAD, LEF, and nuclear β-catenin), molecules of the cell-to-cell adhesion axis (cadherins, catenins), cytoskeletal modulators (Rho family), and extracellular proteases (matrix metalloproteinases, plasminogen activators) (see Thompson et al., Cancer Research 65, 5991-5995, Jul. 15, 2005).

### II. METHODS OF TREATMENT OR PREVENTION OF FIBROTIC DISORDERS

The present invention provides novel methods of treating and/or preventing a fibrotic disorder in a subject. The methods include administering to a subject an effective amount of a BMP9 or BMP10 antagonist, thereby treating and/or preventing a fibrotic disorder in a subject.

The terms “treat,” “treated,” “treating,” and “treatment,” as used herein, refer to the therapeutic measures described herein. The methods of “treatment” include administration of a BMP9 or BMP10 antagonist to a subject in order to cure, reduce the severity of, or ameliorate one or more symptoms of a fibrotic disease or condition, in order to prolong the health or survival of a subject beyond that expected in the absence of such treatment. For example, “treatment” includes the alleviation of a fibrotic disease symptom (e.g., shortness of breath, fatigue, cough, weight loss, loss of appetite associated with pulmonary fibrosis or anorexia, fatigue, weight loss, portal hypertension and ascites associated with liver fibrosis) in a subject by at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or more.

The terms “prevent,” “prevented,” “preventing”, and “prevention,” as used herein refer to the preventative measures described herein. The methods of “prevention” include administration of a BMP9 or BMP10 antagonist to a subject in order to delay, prevent or preclude one or more symptoms of a disease or condition. For example, “prevention” includes a delayed onset or inhibition of a fibrotic disease symptom (e.g., shortness of breath, fatigue, cough, weight loss, loss of appetite associated with pulmonary fibrosis or anorexia, fatigue, weight loss, portal hypertension and ascites associated with liver fibrosis) in a subject by at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or more.

The terms “patient” or “subject” as used herein is intended to include humnan and veterinary patients. In a particular embodiment, the subject is a human. The term “non-human animal” includes all vertebrates, e.g., mammals and non-mammals, such as non-human primates, mice, rabbits, sheep, dog, cow, chickens, amphibians, and reptiles.

A. Indications

The methods of the present invention can be used to treat and/or prevent fibrotic disorders. Exemplary types of fibrotic disorders include, but are not limited to, vascular fibrosis, pulmonary fibrosis (e.g., idiopathic pulmonary fibrosis), pancreatic fibrosis, liver fibrosis (e.g., cirrhosis), renal fibrosis, musculoskeletal fibrosis, cardiac fibrosis (e.g., endomyocardial fibrosis, idiopathic myocardiopathy), skin fibrosis (e.g., scleroderma, post-traumatic, operative cutaneous scarring, keloids and cutaneous keloid formation), eye fibrosis (e.g., glaucoma, sclerosis of the eyes, conjunctival and corneal scarring, and pterygium), progressive systemic sclerosis (PSS), chronic graft versus-host disease, Peyronie's disease, post-cystoscopic urethral stenosis, idiopathic and pharmacologically induced retroperitoneal fibrosis, mediastinal fibrosis, progressive massive fibrosis, proliferative fibrosis, neoplastic fibrosis, Dupuytren's disease, strictures, and radiation induced fibrosis. In a particular embodiment, the fibrotic disorder is not myelofibrosis.

B. Combination Therapies

The present invention contemplates the use of BMP9 and BMP10 antagonists in combination with one or more other therapeutic modalities. Thus, in addition to the use of BMP9 and/or BMP10 antagonists, one may also administer to the subject one or more “standard” therapies for treating fibrotic disorders. For example, the antagonists can be administered in combination with (i.e., together with or linked to (i.e., an immunoconjugate)) cytotoxins, immunosuppressive agents, radiotoxic agents, and/or therapeutic antibodies, Particular co-therapeutics contemplated by the present invention include, but are not limited to, steroids (e.g., corticosteroids, such as Prednisone), immune-suppressing and/or anti-inflammatory agents (e.g., gamma-interferon, cyclophosphamide, azathioprine, methotrexate, penicillamine, cyclosporine, colchicines, antithymocyte globulin, mycophenolate mofetil, and hydroxychloroquine), cytotoxic drugs, calcium channel blockers (e.g., nifedipine), angiotensin converting enzyme inhibitors (ACE) inhibitors, para-aminobenzoic acid (PABA), dimethyl sulfoxide, transforming growth factor-beta (TGF-β) inhibitors, interleukin-5 (IL-5) inhibitors, and pan caspase inhibitors.

Additional anti-fibrotic agents that may be used in combination with BMP9 and/or BMP10 antagonists include, but are not limited to, lectins (as described in, for example, U.S. Pat. No. 7,026,283, the entire contents of which is incorporated herein by reference), as well as the anti-fibrotic agents described by Wynn et al (Journal Clin. Invest. Vol 117 Number 3, March 2007, p524, the entire contents of which is incorporated herein by reference). For example, additional anti-fibrotic agents and therapies include, but are not limited to, various anti-inflammatory/immunosuppressive/cytotoxic drugs (including colchicine, azathioprine, cyclophosphamide, prednisone, thalidomide, pentoxifylline, and theophylline), TGF- [symbol] signaling modifiers (including relaxin, SMAD7, HGF, and BMP7, as well as TGF-β1, TGFβRI, TGFβRII, EGR-1, and CTGF inhibitors), cytokine and cytokine receptor antagonists (inhibitors of IL-1β, IL-5, IL-6, IL-13, IL-21, IL-4R, IL-13Rα1, GM-CSF, TNF-α, oncostatin M, WISP-1, and PDGFs), cytokines and chemokines (IFN-γ, IFN-α/β, IL-12, IL-10, HGF, CXCL10, and CXCL11), chemokine antagonists (inhibitors of CXCL1, CXCL2, CXCL12, CCL2, CCL3, CCL6, CCL17, and CCL18), chemokine receptor antagonists (inhibitors of CCR2, CCR3, CCR5, CCR7, CXCR2, and CXCR4), TLR antagonists (inhibitors of TLR3, TLR4, and TLR9), Angiogenesis antagonists (VEGF-specific antibodies and adenosine deaminase replacement therapy), Antihypertensive drugs (beta blockers and inhibitors of ANG II, ACE, and aldosterone), Vasoactive substances (ET-1 receptor antagonists and bosetan), Inhibitors of the enzymes that synthesize and process collagen (inhibitors of prolyl hydroxylase), B cell antagonists (rituximab), Integrin/adhesion molecule antagonists (molecules that block α1β1 and αvβ6 integrins, as well as inhibitors of integrin linked kinase, and antibodies specific for ICAM-1 and VCAM-1), proapoptotic drugs that target myofibroblasts, MMP inhibitors (inhibitors of MMP2, MMP9, and MMP12), and TIMP inhibitors (antibodies specific for TIMP-1).

The BMP9 and/or BMP10 antagonist and the co-therapeutic agent or co-therapy can be administered in the same formulation or separately. In the case of separate administration, the BMP9 and/or BMP10 antagonist can be administered before, after or concurrently with the co-therapeutic or co-therapy. One agent may precede or follow administration of the other agent by intervals ranging from minutes to weeks. In embodiments where two or more different kinds of therapeutic agents are applied separately to a subject, one would generally ensure that a significant period of time did not expire between the time of each delivery, such that these different kinds of agents would still be able to exert an advantageously combined effect on the target tissues or cells.

In one embodiment, the BMP9 and/or BMP10 antagonist (e.g., an anti-BMP9 or anti-BMP10 antibody) may be linked to a second binding molecule, such as an antibody (i.e., thereby forming a bispecific molecule) or other binding agent that, for example, binds to a different target or a different epitope on BMP9 or BMP10. Examples of additional therapeutic agents that can be used in combination therapy with the antagonists disclosed herein are described in greater detail below in the section on immunoconjugates.

C. Dosages/Amounts

The terms “effective amount” and “therapeutically effective amount” as used herein, refer to that amount of a BMP9 and/or BMP10 antagonist, which is sufficient to effect treatment or prevention of a fibrotic disorder, as described herein, when administered to a subject. A therapeutically effective amount will vary depending upon the subject and the severity of the fibrotic disorder being treated, the weight and age of the subject, the manner of administration and the like, which can readily be determined by one of ordinary skill in the art. The BMP9 and/or BMP10 antagonist dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 mg to about 2,450 mg, about 200 μg to about 2,425 mg, about 300 mg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of a BMP9 and/or BMP10 antagonist. Dosage regimens may be adjusted to provide the optimum therapeutic response. An effective amount is also one in which any toxic or detrimental effects (i.e., side effects) of a BMP9 and/or BMP10 antagonist are minimized and/or outweighed by the beneficial effects.

Actual dosage levels of the BMP9 and/or BMP10 antagonist used in the methods of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular BMP9 and/or BMP10 antagonist employed, or the ester, salt or amide thereof, the route of administration, the time of administration, the rate of excretion of the particular antagonist being employed, the duration of the treatment, other drugs, compounds and/or materials used in combination with the particular antagonist employed, the age, sex, weight, condition, general health and prior medical history of the patient being treated, and like factors well known in the medical arts. A physician or veterinarian having ordinary skill in the art can readily determine and prescribe the effective amount of the antagonist required. For example, the physician or veterinarian could start doses of the antagonist at levels lower than that required in order to achieve the desired therapeutic effect and gradually increase the dosage until the desired effect is achieved. In general, a suitable daily dose of a BMP9 and/or BMP10 antagonist will be that amount which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a BMP9 and/or BMP10 antagonist may be administered as two, three, four, five, six or more sub-doses administered separately at appropriate intervals throughout the day, optionally, in unit dosage forms. While it is possible for a BMP9 and/or BMP10 antagonist of the present invention to be administered alone, it is preferable to administer the antagonist as a pharmaceutical formulation (composition).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the BMP9 and/or BMP10 antagonists used in the methods of the present invention may be administered once or twice weekly by subcutaneous injection or once or twice monthly by subcutaneous injection.

It is especially advantageous to formulate parenteral BMP9 and/or BMP10 antagonists in dosage unit form for ease of administration and uniformity of dosage. Dosage unit form as used herein refers to physically discrete units suited as unitary dosages for the subjects to be treated; each unit contains a predetermined quantity of active antagonist calculated to produce the desired therapeutic effect in association with the required pharmaceutical carrier. The specification for the dosage unit forms are dictated by and directly dependent on (a) the unique characteristics of the active antagonist and the particular therapeutic effect to be achieved, and (b) the limitations inherent in the art of compounding such an active antagonist for the treatment of sensitivity in subjects.

D. Methods of Administration and Formulations

To administer a BMP9 and/or BMP10 antagonist used in the methods of the present invention by certain routes of administration, it may be necessary to include the antagonist in a formulation suitable for preventing its inactivation. For example, the BMP9 and/or BMP10 antagonist may be administered to a subject in an appropriate carrier, for example, liposomes, or a diluent. Pharmaceutically acceptable diluents include saline and aqueous buffer solutions, Liposomes include water-in-oil-in-water CGF emulsions, as well as conventional liposomes (Strejan et al. (1984) J. Neuroimmnunol. 7:27).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active BMP9 and/or BMP10 antagonist, use thereof in a pharmaceutical compositions is contemplated. Supplementary active compounds can also be incorporated with the BMP9 and/or BMP10 antagonist.

Therapeutic BMP9 and/or BMP10 antagonists typically must be sterile and stable under the conditions of manufacture and storage. The antagonist can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including an agent that delays absorption, for example, monostearate salts and gelatin.

Sterile injectable solutions can be prepared by incorporating the active antagonist in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

BMP9 and/or BMP10 antagonists that can be used in the methods of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the antagonist which produces a therapeutic effect. Generally, out of one hundred percent, this amount will range from about 0.001 percent to about ninety percent of active ingredient, preferably from about 0.005 percent to about 70 percent, most preferably from about 0.01 percent to about 30 percent.

The phrases “parenteral administration” and “administered parenterally”, as used herein, means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed along with the BMP9 and/or BMP10 antagonists utilized in the methods of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The BMP9 and/or BMP10 antagonists may also be administered with adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the BMP9 and/or BMP10 antagonists used in the methods of the present invention are administered to humans and animals, they can be given alone or as a pharmaceutical antagonist containing, for example, 0.001 to 90% (more preferably, 0.005 to 70%, such as 0.01 to 30%) of active ingredient in combination with a pharmnaceutically acceptable carrier.

The BMP9 and/or BMP10 antagonists can be administered with medical devices known in the art. For example, in a preferred embodiment, an antagonist can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medications through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, antagonists can be formulated to ensure proper distribution in vivo. For example, the blood-brain barrier (BBB) excludes many highly hydrophilic compounds. To ensure that the BMP9 and/or BMP10 antagonists cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (tee, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antrimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### III. DIAGNOSTIC METHODS

The present invention also provides novel methods for assessing whether a subject has or is at risk of developing a fibrotic disorder. Individuals suspected of having a fibrotic disorder would benefit from early detection, so that fibrotic disease progression can be retarded or even halted. The methods include assessing whether a subject has or is at risk of developing a fibrotic disorder comprising contacting a sample from a subject with a reagent able to detect BMP9 or BMP10 and detecting BMP9 or BMP10, wherein an elevated level of BMP9 or BMP10 relative to a control is an indication that the subject has or is at risk of developing a fibrotic disorder.

The invention further provides methods for determining or predicting the efficacy of a treatment regimen for treating a fibrotic disorder. These methods include assessing the efficacy of a treatment regimen for treating a fibrotic disorder in a subject, the method comprising: a) contacting a first sample obtained from the subject prior to administering at least a portion of the treatment regimen to the subject with a reagent able to detect BMP9 or BMP10; b) contacting a second sample obtained from the subject following administration of at least a portion of the treatment regimen with a reagent table to detect BMP9 or BMP10; and c) comparing the levels of BMP9 or BMP10 from the first and second samples, wherein an elevated level of BMP9 or BMP10 present in the first sample, relative to the second sample, is an indication that the treatment regimen is efficacious for treating a fibrotic disorder in the subject.

In a particular embodiment, the method can include a further step of detecting an additional marker of fibrosis, including, but not limited to, alpha smooth muscle actin, collagen type III, cartilage oligomeric matrix protein, Collagen type I, collagen type IV, fibroblast specific protein-1, fibronectin, serpinE1, periostin, IGFBP3, SPARC, CTGF, TGFb, Cyr61, and phospho smad 2/3, using assays well known in the art. Such assays include, but are not limited to, immunological methods for detection of proteins, protein purification methods, protein function or activity assays, nucleic acid hybridization methods, nucleic acid reverse transcription methods, and nucleic acid amplification methods, ELISA, immunoblotting, Western blotting, Northern blotting, electron microscopy, and Southern blotting. In a particular embodiment, the method can further include detecting fibrosis using biopsies, such as liver biopsies, or commercially available devices, such as Fibroscan (available from London, UK) or Fibrotest (available from France, Europe).

As used herein, the term “sample” includes any body fluid (e.g., blood fluids, lymph, gynecological fluids, cystic fluid, urine, ocular fluids and fluids collected by peritoneal rinsing), or a cell from a subject. Normally, the tissue or cell will be removed form the patient, but in vivo diagnosis is also contemplated. Other patient samples, include tear drops, serum, cerebrospinal fluid, feces, sputum and cell extracts.

As used herein, the term “reagent able to detect BMP9 and/or BMP10” includes any agent capable of binding specifically with BMP9 and BMP10 and transforming BMP9 and/or BMP10 into a detectable moiety. Suitable reagents include antibodies, antibody derivatives, antibody fragments, and the like. Suitable reagents for binding with a BMP9 and/or BMP10 nucleic acid (e.g. a genomic DNA, an mRNA, a spliced mRNA, a cDNA, or the like) include complementary nucleic acids. For example, the nucleic acid reagents may include oligonucleotides (labeled or non-labeled) fixed to a substrate, labeled oligonucleotides not bound with a substrate, pairs of PCR primers, molecular beacon probes, and the like.

As used herein, the term “control” can be the level of BMP9 and/or BMP10 in a sample from a subject not suffering from fibrosis. It can be the same type of sample as the test sample or different. For example, if the sample from the subject being tested is a liver sample (e.g., a cell, a collection of cells, or tissue obtained from a liver biopsy), then the control sample can also be a liver sample from a subject not suffering from a fibrotic disorder. Alternatively, the control sample can be of a different type, e.g., it can be a blood sample from a subject not suffering from a fibrotic disorder. In other embodiments, the control sample can be a collection of samples from a subject not having a fibrotic disorder or a sample from a collection of subjects not have a fibrotic disorder.

As used herein, “an aberrant level” of BMP9 and/or BMP10 is any level of BMP9 and/or BMP10 that differs from the control level of BMP9 and/or BMP10, e.g., significantly higher or elevated levels, or significantly lower or depressed levels.

As used herein, a “higher level,” “elevated level,” or “increased level” of BMP9 and/or BMP10 refers to a level that is elevated relative to a suitable control. Preferably, the differential from the suitable control is greater than the standard error of the assay employed to assess the level. Moreover, the elevated level is preferably at least twice, and more preferably three, four, five, six, seven, eight, nine or ten times the level of BMP9 and/or BMP10 in a suitable control (e.g., a sample from a subject not having a fibrotic disease or the average level of BMP9 and/or BMP10 in several control samples or other suitable benchmark).

As used herein, a “depressed level,” “lower level” or “decreased level” of a BMP9 and/or BMP10 refers to a level that is decreased relative to a suitable control. Preferably, the differential from the suitable control is greater than the standard error of the assay employed to assess the level. The decreased level preferably is at least twice, and more preferably three, four, five, six, seven, eight, nine or ten times lower than the level of the suitable control (e.g., level in a healthy subject not having a fibrotic disease or the average level of BMP9 and/or BMP10 in several control samples or other suitable benchmark).

As used herein, the terms “efficacious” and “efficacy” refers to the likelihood that a treatment regimen will treat a fibrotic disorder in a subject. For example, a treatment regimen is deemed “efficacious” and considered a viable treatment option if the treatment leads to an alleviation of the fibrotic disease symptoms (e.g., shortness of breath, fatigue, cough, weight loss, loss of appetite associated with pulmonary fibrosis or anorexia, fatigue, weight loss, portal hypertension and ascites associated with liver fibrosis) in a subject by at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95% or more.

A. Assays

The presence, absence, and/or level of BMP9 and/or BMP10 in a biological sample obtained from a subject may be assessed by any of a wide variety of in vitro and in vivo techniques and methods. Which transform BMP9 or BMP10 within the sample into a moiety that can be detected and quantified. Non-limiting examples of such methods include analyzing the sample using immunological methods for detection of proteins, protein purification methods, protein function or activity assays, nucleic acid hybridization methods, nucleic acid reverse transcription methods, and nucleic acid amplification methods, enzyme linked immunosorbent assays (ELISAs), immunoblotting, Western blotting, Northern blotting, electron microscopy, mass spectrometry, immunoprecipitations, immunofluorescence, Southern hybridizations and the like. Such techniques, as well as others described herein, can also be used to detect additional fibrotic markers, including, but not limited to alpha smooth muscle actin, collagen type III, cartilage oligomneric matrix protein, Collagen type I, collagen type IV, fibroblast specific protein-1, fibronectin, serpinE1, periostin, IGFBP3, SPARC, CTGF, TGFb, Cyr61, and phospho smad 2/3, where applicable.

In one embodiment, the presence, absence, and/or level BMP9 and/or BMP10 in a sample can be assessed using a reagent, such as an antibody (e.g., a radio-labeled, chromophore-labeled, fluorophore-labeled, or enzyme-labeled antibody), an antibody derivative (e.g., an antibody conjugated with a substrate or with the protein or ligand of a protein-ligand pair (e.g. biotin-streptavidin)), or an antibody fragment (e.g., a single-chain antibody or an isolated antibody hypervariable domain) which binds specifically to and transforms the biomarker, e.g., BMP9 and/or BMP10, in, a sample into a detectable molecule.

The term “labeled”, with regard to the antibody, is intended to encompass direct labeling of the antibody by coupling (i.e., physically linking) a detectable substance to the antibody, as well as indirect labeling of the antibody by reactivity with another reagent that is directly labeled. Examples of indirect labeling include detection of a primary antibody using a fluorescently labeled secondary antibody, such that it can be detected with fluorescently labeled streptavidin.

In another embodiment, the presence, absence, and/or level of BMP9 and/or BMP10 is assessed using a nucleic acid. For example, in one embodiment, the presence, absence, and/or level of BMP9 and/or BMP10 is assessed using a nucleic acid probe.

The term “probe”, as used herein, refers to any molecule that is capable of selectively binding to BMP9 and/or BMP10. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to BMP9 and/or BMP10 mRNA. The nucleic acid probe can be, for example, a full-length cDNA, or a portion thereof such as an oligonucleotide of at least 7, 15, 30, 50, 100, 250 or 500 nucleotides in length and sufficient to specifically hybridize under stringent conditions to BMP9 and/or BMP10 genomic DNA.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in detecting the level of BMP9 and/or BMP10 mRNA.

An alternative method for determining the level of BMP9 and/or BMP10 mRNA in a sample involves the process of nucleic acid amplification, e.g., by RT-PCR (the experimental embodiment set forth in Mullis, 1987, U.S. Pat. No. 4,683,202), ligase chain reaction (Barany (1991) Proc. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, BMP9 and/or BMP10 expression is assessed by quantitative fluorogenic RT-PCR (i.e., the TaqMan™ System). Such methods typically utilize pairs of oligonucleotide primers that are specific for BMP9 and/or BMP10. Methods for designing oligonucleotide primers specific for a known sequence are well known in the art.

The expression levels of BMP9 and/or BMP10 mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of BMP9 and/or BMP10 expression may also comprise using nucleic acid probes in solution.

In one embodiment of the invention, microarrays are used to detect BMP9 and/or BMP10 expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

Furthermore, in vivo techniques for detection of BMP9 and/or BMP10 include introducing into a subject a labeled antibody directed against BMP9 and/or BMP10, which binds to and transforms BMP9 and/or BMP10 into a detectable molecule. As discussed above, the presence, level, or even location of the detectable BMP9 and/or BMP10 in a subject may be detected determined by standard imaging techniques.

In another embodiment, mass spectrometry can be used to detect BMP9 and/or BMP10 in a sample. Mass spectrometry is an analytical technique that consists of ionizing chemical compounds to generate charged molecules (or fragments thereof) and measuring their mass-to-charge ratios. In a typical mass spectrometry procedure, a sample is obtained from a subject, loaded onto the mass spectrometry, and its components (e.g., BMP9 and/or BMP10) are ionized, by different methods (e.g., by impacting them with an electron beam), resulting in the formation of charged particles (ions). The mass-to-charge ratio of the particles is then calculated from the motion of the ions as they transit through electromagnetic fields.

### IV. BMP9 AND BMP10 ANTAGONISTS

As used herein, the term “antagonist” refers to any moiety which downmodulates BMP9 and/or BMP10 activity, including moieties which downregulate BMP9 and/or BMP10 expression or inhibit BMP9 and/or BMP10 function. In one aspect of the invention, the antagonist may be any moiety which directly antagonizes BMP9 and/or BMP10. For example, in one embodiment, the antagonist is a peptide or antibody which binds to BMP9 or BMP10 and prevents BMP9 or BMP10 from binding to its ligand (e.g., activin receptor-like kinase 1 (ALK1), serine/threonine kinase receptors, or endoglin), thereby inhibiting BMP signaling. In another embodiment, the antagonist is a peptide or antibody which binds to the ligand of BMP9 or BMP10 and prevents BMP9 or BMP10 from binding to this ligand. In another aspect of the invention, the moiety indirectly antagonizes BMP9 and/or BMP10 by modulating the activity of downstream mediators in the BMP signaling pathway. In a further aspect of the invention, the antagonist is a receptor-based antagonist, such as a soluble BMP9 or BMP10 receptor, including, but not limited to, soluble ALK1 or serine/threonine kinase receptors.

Representative antagonists, include, but are not limited to, antibodies, nucleic acids (e.g., antisense molecules, such as ribozymes and RNA interfering agents), immunoconjugates (e.g., an antibody conjugated to a therapeutic agent), small molecule inhibitors, fusion proteins, adnectins, aptamers, anticalins, lipocalins, and BMP9 and/or BMP10 derived peptidic compound s.

Particular antagonists contemplated by the present invention, include, but are not limited to, commercially available antibodies, such as MAB3209 (R&D Systems, Minneapolis, Minn. USA) and Alk1-Fc. Additional antagonists contemplated by the present invention include those described in Gazzerro et al. (Rev Endocr MeAtab Disord. 2006; 7:51-65, 23) and Derner, (Clin Podiatr Med. Surg. (2005) 22 607-618), the contents of which are expressly incorporated herein by reference. Specifically, particular antagonists include, but are not limited to, noggin, members of the chordin family, twisted gastrulation, members of the Dan family (e.g., Gremlin, Sclerostin, Dan, uterine sensitization associated gene (USAG-1), Cerberus, Caronte, Coco, protein related to Dan and Cerberus (PRDC) and Dante), nonsignaling membrane BMP pseudoreceptors (e.g., BMP and activin bound protein(BAMBI)), Smad 6, Smad 7 and other intracellular BMP antagonists.

A. Antibodies

In one embodiment of the invention, the therapeutic and diagnostic methods described herein employ an antibody that binds, e.g., directly to or indirectly to, and inhibits BMP9 or BMP10 activity and/or down-modulates BMP9 or BMP10 expression.

i. General

The term “antibody” or “immunoglobulin,” as used interchangeably herein, includes whole antibodies and any antigen binding fragment (i.e., “antigen-binding portion”) or single chains thereof. An “antibody” comprises at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., BMP9 and/or BMP10). It has been shown that the antigen-binding function of an antibody can be performed by fragments of a full-length antibody. Examples of binding fragments encompassed within the term “antigen-binding portion” of an n antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb including VH and VL domains; (vi) a dAb fragment (Ward et al (1989) Nature 341, 544-546), which consists of a V_{H} domain; (vii) a dAb which consists of a VH or a VL domain; and (viii) an isolated complementarity determining region (CDR) or (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L}, and V_{H} regions pair to form monovalent molecules (known as single chain Fv (scFv); see e.g., Bird et al. (1988) Science 242, 423-426; and Huston et al. (1988) Proc. Natl. Acad. Sci. USA 85, 5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies can be prepared using any art recognized technique and those described herein such as, for example, a hybridoma method, as described by Kohler et al. (1975) Nature, 256:495, a trausgenic animal, as described by, for example, (see e.g., Lonberg, et al. (1994) Nature 368(6474): 856-859), recombinant DNA methods (see, e.g., U.S. Pat. No. 4,816,567), or using phage antibody libraries using the techniques described in, for example, Clackson et al., Nature, 352:624-628 (1991) and Marks et al., J. Mol. Biol., 222:581-597 (1991). Monoclonal antibodies include chimeric antibodies, human antibodies and humanized antibodies and may occur naturally or be recombinantly produced.

The term “recombinant antibody,” refers to antibodies that are prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain embodiments, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The ten “human antibody,” as used herein, is intended to include antibodies having variable regions in which both the framework and CDR regions are derived from human germline immunoglobulin sequences as described, for example, by Kabat et al. (See Kab at, et al. (1991) Sequences of proteins of Immunological Interest, Fifth Edition, U.S. Department of Health and Human Services, NIH Publication No. 91-3242). Furthermore, if the antibody contains a constant region, the constant region also is derived from human germline immunoglobulin sequences. The human antibodies may include amino acid residues not encoded by human germline immunoglobulin sequences (e.g. mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The human antibody can have at least one or more amino acids replaced with an amino acid residue, e.g., an activity enhancing amino acid residue which is not encoded by the human gremlin immunoglobulin sequence. Typically, the human antibody can have up to twenty positions replaced with amino acid residues which are not part of the human germline immunoglobulin sequence. In a particular embodiment, these replacements are within the CDR regions as described in detail below.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

A “bispcific” or “bifunctional antibody” is an artificial hybrid antibody having two different heavy/light chain pairs and two different binding sites. Bispecific antibodies can be produced by a variety of methods including fusion of hybridomas or linking of Fab′ fragments, See, e.g., Songsivilai & Lachmann, (1990) Clin. Exp. Immunol. 79, 315-321; Kostelny et al. (1992) J. Immunol. 148, 1547-1553.

As used herein, a “heterologous antibody” is defined in relation to the transgenic non-human organism or plant producing such an antibody.

An “isolated antibody,” as used herein, is intended to refer to an antibody which is substantially free of other antibodies having different antigenic specificities (e.g., an isolated antibody that specifically binds to BMP9 or BMP10 is substantially free of antibodies that specifically bind antigens other than BMP9 or BMP10). In addition, an isolated antibody is typically substantially free of other cellular material and/or chemicals. In one embodiment of the invention, a combination of “isolated” monoclonal antibodies having different binding specificities are combined in a well defined composition.

As used herein, “isotype” refers to the antibody class (e.g., IgM or IgG1) that is encoded by heavy chain constant region genes. In one embodiment, an antibody or antigen binding portion thereof is of an isotype selected from an IgG1, an IgG2, an IgG3, an IgG4, an IgM, an IgA1, an TgA2, an IgAsec, an IgD, or an IgE antibody isotype.

As used herein, “isotype switching” refers to the phenomenon by which the class, or isotype, of an antibody changes from one Ig class to one of the other Ig classes.

As used herein, “nonswitched isotype” refers to the isotypic class of heavy chain that is produced when no isotype switching has taken place; the CH gene encoding the nonswitched isotype is typically the first CH gene immediately downstream from the functionally rearranged VDJ gene. Isotype switching has been classified as classical or non-classical isotype switching. Classical isotype switching occurs by recombination events which involve at least one switch sequence regions in a gene encoding an antibody. Non-classical isotype switching may occur by, for example, homologous recombination between human σ_{μ} and human Σ_{μ} (δ-associated deletion). Alternative non-classical switching mechanisms, such as intertransgene and/or interchronmosomal recombination, among others, may occur and effectuate isotype switching.

As used herein, the term “switch sequence” refers to those DNA sequences responsible for switch recombination. A “switch donor” sequence, typically a μ switch region, will be 5′ (i.e., upstream) of the construct region to be deleted during the switch recombination. The “switch acceptor” region will be between the construct region to be deleted and the replacement constant region (e.g., γ, ε, etc.). As there is no specific site where recombination always occurs, the final gene sequence will typically not be predictable from the construct.

The term “epitope” or “antigenic determinant” refers to a site on an antigen to which an ilmmunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art and those described herein, for example, x-ray crystallography and 2-dimensional nuclear magnetic resonance. See, e.g., Epitope Mapping Protocols in Methods in Molecular Biology, Vol. 66, G. E. Morris, Ed. (1996).

Antibody proteins obtained from members of the camel and dromedary (Camelus bactrianus and Calelus dromrnaderius) family, including New World members such as Ilama species (Lama paccos, Lama glama and Lama vicugna), have been characterized with respect to size, structural complexity and antigenicity for human subjects. Certain IgG antibodies found in nature in this family of mammals lack light chains, and are thus structurally distinct from the four chain quaternary structure having two heavy and two light chains typical for antibodies from other animals. See, for example, PCT Publication WO 94/04678.

A region of the camelid antibody that is the small, single variable domain identified as Vim can be obtained by genetic engineering to yield a small protein having high affinity for a target, resulting in a low molecular weight, antibody-derived protein known as a “camelid nanobody”. See U.S. Pat. No. 5,759,808; see also Stijlemans et al., 2004 J. Biol. Chem. 279: 1256-1261; Dumoulin et al., 2003 Nature 424: 783-788; Pleschbgeger et al., 2003 Bioconjugate Chem. 14: 440-448; Cortez-Retamozo et al., 2002 Int. J. Cancer 89: 456-62; and Lauwereys. et al, 1998 EMBO J. 17: 3512-3520. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camnelid nanobodies are useful as reagents to detect antigens that are otherwise cryptic using classical immunological techniques, and as possible therapeutic agents. Thus, yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies' being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitate drug transport across the blood brain barrier. See U.S. Pat. Pub. No, 20040161738, published. Aug. 19, 2004. These features combined with the low antigenicity in humans indicate great therapeutic potential. Further, these molecules can be fully expressed in prokaryotic cells such as E. coli.

Accordingly, a feature of the present invention is a camelid antibody or camelid nanobody having high affinity for BMP9 or BMP10. In certain embodiments herein, the camelid antibody or nanobody is naturally produced in the camelid animal, i.e., is produced by the camelid following immunization with BMP9 or BMP10 or a peptide fragment thereof, using techniques described herein for other antibodies. Alternatively, the camelid nanobody is engineered, i.e., produced by selection, for example from a library of phage displaying appropriately mutagenized camelid nanobody proteins using panning procedures. Engineered nanobodies can further be customized by genetic engineering to increase the half life in a recipient subject from 45 minutes to two weeks.

Diabodies are bivalent, bispecific molecules in which V_{H} and V_{L} domains are expressed on a single polypeptide chain, connected by a linker that is too short to allow for pairing between the two domains on the same chain. The V_{H} and V_{L} domains pair with complementary domains of another chain, thereby creating two antigen binding sites (see e.g., Holliger et al., 1993 Proc. Natl. Acad. Sci USA 90:6444-6448; Poljak et al., 1994 Structure 2:1121-1123). Diabodies can be produced by expressing two polypeptide chains with either the structure V_{HA}-V_{LB} and V_{HB}-V_{LA} (V_{H}-V_{L} configuration), or V_{LA}-V_{HB} and V_{LB}-V_{HA} (V_{L}-V_{H} configuration) within the same cell. Most of them can be expressed in soluble form in bacteria.

Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack., 1997 Immunotechnology, 3(2): 83-105; Ridgway et al., 1996 Protein Eng., 9(7):617-21).

A diabody can be fused to Fc to generate a “di-diabody” (see Lu at al., 2004 J. Biol. Chem., 279(4):2856-65).

The invention further provides BMP9 or BMP10 binding molecules that exhibit functional properties of antibodies but derive their framework and antigen binding portions from other polypeptides (e.g., polypeptides other than those encoded by antibody genes or generated by the recombination of antibody genes in vivo). The antigen binding domains (e.g., BMP9 or BMP10 binding domains) of these binding molecules are generated through a directed evolution process. See U.S. Pat. No. 7,115,396. Molecules that have an overall fold similar to that of a variable domain of an antibody (an “immunoglobulin-like” fold) are appropriate scaffold proteins. Scaffold proteins suitable for deriving antigen binding molecules include fibronectin or a fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MUC, T-cell antigen receptor, CD1, C2 and I-set domains of VCAM-1, I-set immunoglobulin domain of myosin-binding protein C, I-set immunoglobulin domain of myosin-binding protein H, I-set immunoglobulin domain of telokin, NCAM. twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

The antigen binding domain (e.g., the immunoglobulin-like fold) of the non-antibody binding molecule can have a molecular mass less than 10 kD or greater than 7.5 kD (e.g., a molecular mass between 7.5-10 kD). The protein used to derive the antigen binding domain is a naturally occurring mammalian protein (e.g., a human protein), and the antigen binding domain includes up to 50% (e.g., up to 34%, 25%, 20%, or 15%), mutated amino acids as compared to the immunoglobulin-like fold of the protein from which it is derived. The domain having the immunoglobulin-like fold generally consists of 50-150 amino acids (e.g., 40-60 amino acids).

To generate non-antibody binding molecules, a library of clones is created in which sequences in regions of the scaffold protein that form antigen binding surfaces (e.g., regions analogous in position and structure to CDRs of an antibody variable domain immunoglobulin fold) are randomized. Library clones are tested for specific binding to the antigen of interest (e.g., BMP9 or BMP10) and for other functions (e.g., inhibition of biological activity of BMP9 or BMP10). Selected clones can be used as the basis for further randomization and selection to produce derivatives of higher affinity for the antigen.

High affinity binding molecules are generated, for example, using the tenth module of fibronectin III (¹⁰Fn3) as the scaffold. A library is constructed for each of three CDR-like loops of ¹⁰FN3 at residues 23-29, 52-55, and 78-87. To construct each library, DNA segments encoding sequence overlapping each CDR-like region are randomized by oligonucleotide synthesis. Techniques for producing selectable ¹⁰Fn3 libraries are described in U.S. Pat. Nos. 6,818,418 and 7,115,396; Roberts and Szostak, 1997 Proc. Natl. Acad. Sci. USA 94:12297; U.S. Pat. No. 6,261,804; U.S. Pat. No. 6,258,558; and Szostak et al. WO98/31700.

Non-antibody binding molecules can be produces as dimers or multimers to increase avidity for the target antigen. For example, the antigen binding domain is expressed as a fusion with a constant region (Fc) of an antibody that forms Fc-Fc dimers, See, e.g., U.S. Pat. No. 7,115,396.

Antibodies that can be used in the methods of the present invention also include those antibodies that bind the same or an overlapping epitope as the particular antibodies described herein, i.e., antibodies that compete for binding to BMP9 or BMP10, or bind to an epitope on BMP9 or BMP10 recognized by the particular antibodies described herein.

Antibodies that recognize the same or an overlapping epitope can be identified using routine techniques such as an immunoassay, for example, by showing the ability of one antibody to block the binding of another antibody to a target antigen, i.e., a competitive binding assay. Competitive binding is determined in an assay in which the immunoglobulin under test inhibits specific binding of a reference antibody to an antigen, such as BMP9 or BMP10. Numerous types of competitive binding assays are known, for example: solid phase direct or indirect radioimmunoassay (RIA), solid phase direct or indirect enzyme immunoassay (EIA), sandwich competition assay (see Stahli et al., (1983) Methods in Enzymology 9:242); solid phase direct biotin-avidin EIA (see Kirkland et al., (1986) J. Immunol. 137:3614); solid phase direct labeled assay, solid phase direct labeled sandwich assay (see Harlow and Lane, (1988) Antibodies: A Laboratry Manual, Cold Spring Harbor Press); solid phase direct label RIA using I-125 label (see Morel et al., (1988) Mol. Immunol. 25(1):7); solid phase direct biotin-avidin EIA (Cheung et al., (1990) Virology 176:546); and direct labeled RIA. (Moldenhauer et al., (1990) Scand. J. Immunol. 32:77). Typically, such an assay involves the use of purified antigen (e.g., BMP9 or BMP10) bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

As used herein, the terms “specific binding,” “specifically binds,” “selective binding,” and “selectively binds,” mean that an antibody or antigen-binding portion thereof, exhibits appreciable affinity for a particular antigen or epitope and, generally, does not exhibit significant cross-reactivity with other antigens and epitopes. “Appreciable” or preferred binding includes binding with an affinity of at least 10⁶, 10⁷, 10⁸, 10⁹ M⁻¹, or 10¹⁰ M⁻¹. Affinities greater than 10⁷ M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross-reactivity” is one that will not appreciably bind to an undesirable entity (e.g., an undesirable proteinaceous entity). Specific or selective binding can be determined according to any art-recognized means for determining such binding, including, for example, according to Scatchard analysis and/or competitive binding assays.

The term “K_{D}” as used herein, is intended to refer to the dissociation equilibrium constant of a particular antibody-antigen interaction or the affinity of an antibody for an antigen. In one embodiment, the antibody or antigen binding portion thereof according to the present invention binds an antigen (e.g., BMP9 or BMP10) with an affinity (K_{D}) of 50 nM or better (i.e., or less) (e.g., 40 nM or 30 nM or 20 nM or 10 nM or less), as measured using a surface plasmon resonance assay or a cell binding assay. In a particular embodiment, an antibody or antigen binding portion thereof according to the present invention binds BMP9 or BMP10 with an affinity (K_{D}) of 8 nM or better (e.g., 7 nM, 6 nM, 5 nM, 4 nM, 2 nM, 1.5 nM, 1.4 nM, 1.3 nM, 1 nM or less), as measured by a surface plasmon resonance assay or a cell binding assay. In other embodiments, an antibody or antigen binding portion thereof binds an antigen (e.g., BMP9 or BMP10) with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 3000 instrument using recombinant BMP9 or BMP10 as the analyte and the antibody as the ligand, and binds to the predetermined antigen with an affinity that is at least two-fold greater than its affinity for binding to a non-specific antigen (e.g., BSA, casein) other than the predetermined antigen or a closely-related antigen.

The term “K_{off},” as used herein, is intended to refer to the off rate constant for the dissociation of an antibody from the antibody/antigen complex.

The term “EC50,” as used herein, refers to the concentration of an antibody or an antigen-binding portion thereof, which induces a response, either in an in vitro or an in vivo assay, which is 50% of the maximal response, i.e., halfway between the maximal response and the baseline.

As used herein, “glycosylation pattern” is defined as the pattern of carbohydrate units that are covalently attached to a protein, more specifically to an immunoglobulin protein.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “rearranged” as used herein refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus will have at least one recombined heptamer/nonamer homology element.

The term “unrearranged” or “germline configuration” as used herein in reference to a V segment refers to the configuration wherein the V segment is not recombined so as to be immediately adjacent to a D or J segment.

The term “modifying,” or “modification,” as used herein, is intended to refer to changing one or more amino acids in the antibodies. The change can be produced by adding, substituting or deleting an amino acid at one or more positions. The change can be produced using known techniques, such as PCR mutagenesis. For example, in some embodiments, an antibody employed by the methods of the present invention can be modified, to thereby modify the binding affinity of the antibody to BMP9 or BMP10.

The present invention also encompasses “conservative amino acid substitutions” in the sequences of the antibodies used in the methods of the invention, i.e., nucleotide and amino acid sequence modifications which do not abrogate the binding of the antibody encoded by the nucleotide sequence or containing the amino acid sequence, to the antigen, i.e., BMP9 or BMP10. Conservative amino acid substitutions include the substitution of an amino acid in one class by an amino acid of the same class, where a class is defined by common physicochemical amino acid side chain properties and high substitution frequencies in homologous proteins found in nature, as determined, for example, by a standard Dayhoff frequency exchange matrix or BLOSUM matrix. Six general classes of amino acid side chains have been categorized and include: Class I (Cys); Class II (Ser, Ttr, Pro, Ala, Oly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution. Thus, a predicted nonessential amino acid residue in an anti-BMP9 or BMP10 antibody of the present invention is preferably replaced with another amino acid residue from the same class. Methods of identifying nucleotide and amino acid conservative substitutions which do not eliminate antigen binding are well-known in the art (see, e.g. Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

The term “non-conservative amino acid substitution” refers to the substitution of an amino acid in one class with an amino acid from another class; for example, substitution of an Ala, a class II residue, with a class III residue such as Asp, Asn, Glu, or Gln.

Alternatively, in another embodiment, mutations (conservative or non-conservative) can be introduced randomly along all or part of an anti-BMP9 or BMP10 antibody coding sequence, such as by saturation mutagenesis, and the resulting modified anti-BMP9 or BMP10 antibodies can be screened for binding activity.

A “consensus sequence” is a sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” of an immunoglobulin refers to a framework region in the consensus immunoglobulin sequence.

ii. Engineered and Modified Antibodies

An antibody of the invention can be prepared using an antibody having one or more V_{H} and/or V_{H} sequences as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain CDRs. For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., 1998 Nature 332:323-327; Jones et al., 1986 Nature 321:522-525; Queen et al., 1989 Proc. Natl. Acad. See. U.S.A. 86:10029-10033; U.S. Pat. No. 5,225,539, and U.S. Pat. Nos. 5,530,101; 5,585,089; 5,693,762 and 6,180,370).

Framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kahat et al., 1991 Sequences of Proteins of Immunological Interest, Fifth Edition, U.S. Department of Health and Human Services, NIH Publication No. 91-3242; Tomlinson et al., 1992 J. Mol. Biol. 227:776-798; and Cox et al., 1994 Eur. J. Immunol. 24:827-836; the contents of each of which are expressly incorporated herein by reference.

The V_{H} CDR1, 2 and 3 sequences and the V_{L} CDR1, 2 and 3 sequences can be grafted onto framework regions that have the identical sequence as that found in the germline immunoglobulin gene from which the framework sequence is derived, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

CDRs can also be grafted into framework regions of polypeptides other than immunoglobulin domains. Appropriate scaffolds form a conformationally stable framework that displays the grafted residues such that they form a localized surface and bind the target of interest (e.g., BMP9 or BMP10). For example, CDRs can be grafted onto a scaffold in which the framework regions are based on fibronectin, ankyrin, lipocalin, neocarzinostain, cytochrome b, CP1 zinc finger, PST1, coiled coil, LACI-D1, Z domain or tendramisat (See e.g., Nygren and Uhlen, 1997 Current Opinion in Structural Biology, 7, 463-469).

Another type of variable region modification is mutation of amino acid residues within the V_{H} and/or V_{L} CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s), and the effect on antibody binding, or other functional property of interest, can be evaluated in in vitro or in vivo assays as described herein. Conservative modifications can be introduced. The mutations may be amino acid substitutions, additions or deletions. Moreover, typically no more than one, two, three, four or five residues within a CDR region are altered.

Engineered antibodies of the invention include those in which modifications have been made to framework residues within V_{H} and/or V_{L}, e.g., to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Pat. Pub. No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, U.S. Pat. No. 6,277,375 describes the following mutations in an IgG that increase its half-life in vivo: T252L, T254S, T256F. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121.022 by Presta et al.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the antibody to fix complement. This approach is described further in WO 94/29351 by Bodmer et al.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in WO 00/42072 by Presta. Moreover, the binding sites on human IgG1 for FcγRI, FcγRII, FcγRIII and FcRn have been mapped and variants with improved binding have been described (see Shields, R. L. et al., 2001 J. Biol. Chem. 276:6591-6604).

In still another embodiment, the glycosylation of an antibody is modified. For example, an aglycoslated antibody can be made (i.e., the antibody lacks glycosylation). Glycosylation can be altered, for example, to increase the affinity of the antibody for an antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Pub. WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). WO 99/54342 by Umana et al. describes cell lines engineered to express glycoprotein-modifying glycosyl transferases (e.g., beta(1,4)—N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Another modification of the antibodies herein that is contemplated by the invention is pegylation. An antibody can be pegylated to, for example, increase the biological (e.g. serum) half-life of the antibody. To pegylate an antibody, the antibody, or fragment thereof, typically is reacted with polyethylene glycol (PEG), such as a reactive ester or aldehyde derivative of PEG, under conditions in which one or more PEG moieties become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et a.

In addition, pegylation can be achieved in any part of a BMP9 or BMP10 binding polypeptide of the invention by the introduction of a nonnatural amino acid. Certain nonnatural amino acids can be introduced by the technology described in Deiters et al., J Am Chem Soc 125:11782-11783, 2003: Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. The polypeptides containing these novel amino acids can then be pegylated at these chosen, sites in the protein.

iii. Antibody Fragments and Antibody Mimetics

The instant invention is not limited to traditional antibodies and may be practiced through the use of antibody fragments and antibody mimetics. As detailed below, a wide variety of antibody fragment and antibody mimetic technologies have now been developed and are widely known in the art. While a number of these technologies, such as domain antibodies, Nanobodies, and UniBodies make use of fragments of, or other modifications to, traditional antibody structures, there are also alternative technologies, such as Adnectins, Affibodies, DARPins, Anticalins, Avimers, and Versabodies that employ binding structures that, while they mimic traditional antibody binding, are generated from and function via distinct mechanisms. Some of these alternative structures are reviewed in Gill and Damle (2006) 17: 653-658.

Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, domain antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; U.S. Ser. No. 2004/0110941; European patent application No. 1433846 and European Patents 0368684 & 0616640; WO05/035572, WO04/101790, WO04/081026, WO04/058821, WO004/003019 and WO03/002609, each of which is herein incorporated by reference in its entirety.

Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harbouring the full antigen-binding capacity of the original heavy-chain antibody. Nanobodies have a high homology with the VH domains of human antibodies and can be further humanized without any loss of activity. Importantly, Nanobodies have a low immunogenic potential, which has been confinned in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see, e.g., WO 04/041867, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see, e.g., WO 06/079372, which is herein incorporated by reference in its entirety) is a proprietary method for generating Nanobodies against a desired target, based on automated high-throughout selection of B-cells and could be used in the context of the instant invention.

UniBodies are another antibody fragment technology, however this one is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, UniBfodies may function to inhibit or silence, but not kill, the cells to which they are bound. Additionally, UniBody binding to cancer cells do not stimulate them to proliferate. Furthermore, because UniBodies are about half the size of traditional IgG4 antibodies, they may show better distribution over larger solid tumors with potentially advantageous efficacy. UniBodies are cleared from the body at a similar rate to whole IgG4 antibodies and are able to bind with a similar affinity for their antigens as whole antibodies. Further details of UniBodies may be obtained by reference to patent application WO2007/059782, which is herein incorporated by reference in its entirety.

Adnectin molecules are engineered binding proteins derived from one or more domains of the fibronectin protein. Fibronectin exists naturally in the human body. It is present in the extracellular matrix as an insoluble glycoprotein dimer and also serves as a linker protein. It is also present in soluable form in blood plasma as a disulphide linked dimer. The plasma form of fibronectin is synthesized by liver cells (hepatocytes), and the ECM form is made by chondrocytes, macrophages, endothelial cells, fibroblasts, and some cells of the epithelium (see Ward M., and Marcey, D., callutheran.edu/Academic_ProgramsDepartments/BioDev/omm/fibro/fibro.htm). As mentioned previously, fibronectin may function naturally as a cell adhesion molecule, or it may mediate the interaction of cells by making contacts in the extracellular matrix. Typically, fibronectin is made of three different protein modules, type I, type II, and type III modules. For a review of the structure of function of the fibronectin, see Pankov and Yamada (2002) J Cell Sci.; 115(Pt 20):3861-3, Hohenester and Engel (2002) 21:115-128, and Lucena et al. (2007) Invest Clin. 48:249-262.

In a preferred embodiment, adnectin molecules are derived from the fibronectin type III domain by altering the native protein which is composed of multiple beta strands distributed between two beta sheets. Depending on the originating tissue, fibronecting may contain multiple type III domains which may be denoted, e.g., ¹Fn3, ²Fn3, ³Fn3, etc. The ¹⁰Fn3 domain contains an integrin binding motif and further contains three loops which connect the beta strands. These loops may be thought of as corresponding to the antigen binding loops of the IgG heavy chain, and they may be altered by methods discussed below to specifically bind a target of interest, e.g., BMP9 or BMP10. Preferably, a fibronectin type III domain useful for the purposes of this invention is a sequence which exhibits a sequence identity of at least 30%, at least 40%, at least 50%, at least 60%, at least 70%, at least 80%, at least 90%, or at least 95% to the sequence encoding the structure of the fibronectin type III molecule which can be accessed from the Protein Data Bank (PDB, resb.org/pdb/home/home.do) with the accession code: 1 ttg. Adnectin molecules may also be derived from polymers of ¹⁰Fn3 related molecules rather than a simple monomeric ¹⁰Fn3 structure.

Although the native ¹⁰Fn3 domain typically binds to integrin, ¹⁰Fn3 proteins adapted to become adnectin molecules are altered so to bind antigens of interest, e.g., BMP9 or BMP10. In one embodiment, the alteration to the ¹⁰Fn3 molecule comprises at least one mutation to a beta strand. In a preferred embodiment, the loop regions which connect the beta strands of the ¹⁰Fn3 molecule are altered to bind to an antigen of interest, e.g., BMP9 or BMP10.

The alterations in the ¹⁰Fn3 may be made by any method known in the art including, but not limited to, error prone PCR, site-directed mutagenesis, DNA shuffling, or other types of recombinational mutagenesis which have been referenced herein. In one example, variants of the DNA encoding the ¹⁰Fn3 sequence may be directly synthesized in vitro, and later transcribed and translated in vitro or in vivo. Alternatively, a natural ¹⁰Fn3 sequence may be isolated or cloned from the genome using standard methods (as performed, e.g., in U.S. Pat. Application No. 20070082365), and then mutated using mutagenesis methods known in the art.

In one embodiment, a target protein, e.g., BMP9 or BMP10, may be immobilized on a solid support, such as a column resin or a well in a microtiter plate. The target is then contacted with a library of potential binding proteins. The library may comprise ¹⁰Fn3 clones or adnectin molecules derived from the wild type ¹⁰Fn3 by mutagenesis/randomization of the ¹⁰Fn3 sequence or by mutagenesis/randomization of the ¹⁰Fn3 loop regions (not the beta strands). In a preferred embodiment the library may be an RNA-protein fusion library generated by the techniques described in Szostak et al., U.S. Ser. No. 09/007,005 and 09/247,190; Szostak et al, WO989/31700; and Roberts & Szostak (1997) 94:12297-12302. The library may also be a DNA-protein library (e.g., as described in Lohse, U.S. Ser. No. 60/110,549, U.S. Ser. No. 09/459,190, and WO 00/32823). The fusion library is then incubated with the immobilized target (e.g., BMP9 or BMP10) and the solid support is washed to remove non-specific binding moieties. Tight binders are then eluted under stringent conditions and PCR is used to amply the genetic information or to create a new library of binding molecules to repeat the process (with or without additional mutagenesis). The selection/mutagenesis process may be repeated until binders with sufficient affinity to the target are obtained. Adnectin molecules for use in the present invention may be engineered using the PROfusion™ technology employed by Adnexus, a Briston-Myers Squibb company. The PROfusion technology was created, based on the techniques referenced above (e.g., Roberts & Szostak (1997) 94:12297-12302). Methods of generating libraries of altered ¹⁰Fn3 domains and selecting appropriate binders which may be used with the present invention are described fully in the following U.S. patent and patent application documents and are incorporated herein by reference: U.S. Pat. Nos. 7,115,396; 6,818,418; 6,537,749; 6,660,473; 7,195,880; 6,416,950; 6,214,553; 6,623,926; 6,312,927; 6,602,685; 6,518,018; 6,207,446; 6,258,558; 6,436,665; 6,281,344; 7,270,950; 6,951,725; 6,846,655; 7,078,197; 6,429,300; 7,125,669; 6,537,749; 6,660,473; and U.S. Pat. Application Nos. 20070082365; 20050255548; 20050038229; 20030143616; 20020182597; 20020177158; 20040086980; 20040253612; 20030022236; 20030013160; 20030027194; 20030013110; 20040259155; 20020182687; 20060270604; 20060246059; 20030100004; 20030143616; and 20020182597. The generation of diversity in fibronectin type III domains, such as ¹⁰Fn3, followed by a selection step may be accomplished using other methods known in the art such as phage display, ribosome display, or yeast surface display, e.g., Lipov\check{s}ek et al. (2007) Journal of Molecular Biology 368: 1024-1041; Sergeeva et al. (2006) Adv Drug Deliv Rev 58:1622-1654; Petty et al. (2007) Trends Biotechnol. 25: 7-15; Rothe et al (2006) Expert Opin Biol Ther. 6:177-187; and Hoogenboom (2005) Nat. Biotechnol. 23:1105-1116.

It should be appreciated by one of skill in the art that the methods references cited above may be used to derive antibody mimics from proteins other than the preferred ¹⁰Fn3 domain. Additional molecules which can be used to generate antibody mimics via the above referenced methods include, without limitation, human fibronectin modules ¹Fn3-⁹Fn3 and ¹¹Fn3-¹⁷Fn3 as well as related Fn3 modules from non-human animals and prokaryotes. In addition, Fn3 modules from other proteins with sequence homology to ¹⁰Fn3, such as tenascins and undulins, may also be used. Other exemplary proteins having immunoglobulin-like folds (but with sequences that are unrelated to the V_{H} domain) include N-cadherin, ICAM-2, titin, GCSF receptor, cytokine receptor, glycosidase inhibitor, E-cadherin, and antibiotic chromoprotein. Further domains with related structures may be derived from myelin membrane adhesion molecule P0, CD8, CD4, CD2, class I MHC, T-cell antigen receptor, CD1, C2 and I-set domains of VCAM-1, I-set immunoglobulin fold of myosin-binding protein C, I-set immunoglobulin fold of myosin-binding protein H, I-set immunoglobulin-fold of telokin, telikin, NCAM, twitchin, neuroglian, growth hormone receptor, erythropoietin receptor, prolactin receptor, GC-SF receptor, interferon-gamma receptor, beta-galactosidase/glucuronidase, beta-glucuronidase, and transglutaminase. Alternatively, any other protein that includes one or more immunoglobulin-like folds may be utilized to create a adnecting like binding moiety. Such proteins may be identified, for example, using the program SCOP (Murzin et al., J. Mol. Biol. 247:536 (1995); Lo Conte et al., Nucleic Acids Res. 25:257 (2000).

An aptamer is another type of antibody-mimetic which is encompassed by the present invention. Aptamers are typically small nucleotide polymers that bind to specific molecular targets. Aptamers may be single or double stranded nucleic acid molecules (DNA or RNA), although DNA based aptamers are most commonly double stranded. There is no defined length for an aptamer nucleic acid; however, aptamer molecules are most commonly between 15 and 40 nucleotides long.

Aptamers often form complex three-dimensional structures which determine their affinity for target molecules. Aptamers can offer many advantages over simple antibodies, primarily because they can be engineered and amplified almost entirely in vitro. Furthermore, aptamers often induce little or no immune response.

Aptamers may be generated using a variety of techniques, but were originally developed using in vitro selection (Ellington and Szostak. (1990) Nature. 346(6287):818-22) and the SELEX method (systematic evolution of ligands by exponential enrichment) (Schneider et al. 1992. J Mol. Biol. 228(3):862-9) the contents of which are incorporated herein by reference. Other methods to make and uses of aptamers have been published including Klussmann. The Aptamer Handbook: Functional Oligonucleotides and Their Applications. ISBN: 978-3-527-31059-3; Ulrich et al. 2006. Comb Chem High Throughput Screen 9(8):619-32; Cerchia and de Franciscis. 2007. Methods Mol. Biol. 361:187-200; Ireson and Kelland. 2006. Mol Cancer Ther. 2006 5(12):2957-62; U.S. Pat. Nos. 5,582,981; 5,840,867; 5,756,291; 6,261,783; 6,458,559; 5,792,613; 6,111,095; and U.S. patent application Ser. Nos. 11/482,671; 11/102,428; 11/291,610; and 10/627,543 which are all incorporated herein by reference.

The SELEX method is clearly the most popular and is conducted in three fundamental steps. First, a library of candidate nucleic acid molecules is selected from for binding to specific molecular target. Second, nucleic acids with sufficient affinity for the target are separated from non-binders. Third, the bound nucleic acids are amplified, a second library is formed, and the process is repeated. At each repetition, aptamers are chosen which have higher and higher affinity for the target molecule. SELEX methods are described more fully in the following publications, which are incorporated herein by reference: Bugaut et al. 2006, 4(22):4082-8; Stoltenburg et al. 2007 Biomol Eng. 2007 24(4):381-403; and Gopinath. 2007. Anal Bioanal Chem. 2007, 387(1):171-82.

An “aptamer” of the invention also been includes aptamer molecules made from peptides instead of nucleotides. Peptide aptamers share many properties with nucleotide aptamers (e.g., small size and ability to bind target molecules with high affinity) and they may be generated by selection methods that have similar principles to those used to generate nucleotide aptamers, for example Baines and Colas. 2006. Drug Discov Today. 11(7-8):334-41; and Bickle et al. 2006. Nat. Protoc. 1(3):1066-91 which are incorporated herein by reference.

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55). The simple, robust structure of Affibody molecules in combination with their low molecular weight (6 kDa), make them suitable for a wide variety of applications, for instance, as detection reagents (Romnark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self compatible repeats displaying variable surface residues and their random assembly into repeat domains.

DARPins can be produced in bacterial expression systems at very high yields and they belong to the most stable proteins known. Highly specific, high-affinity DARPins to a broad range of target proteins, including human receptors, cytokines, kinases, human proteases, viruses and membrane proteins, have been selected. DARPins having affinities in the single-digit nanomolar to picomolar range can be obtained.

DARPins have been used in a wide range of applications, including ELISA, sandwich ELISA, flow cytometric analysis (FACS), immunohistochemistry (IHC), chip applications, affinity purification or Western blotting, DARPins also proved to be highly active in the intracellular compartment for example as intracellular marker proteins fused to green fluorescent protein (GFP). DARPins were further used to inhibit viral entry with IC50 in the pM range. DARPins are not only ideal to block protein-protein interactions, but also to inhibit enzymes. Proteases, kinases and transporters have been successfully inhibited, most often an allosteric inhibition mode. Very fast and specific enrichments on the tumor and very favorable tumor to blood ratios make DARPins well suited for in vivo diagnostics or therapeutic approaches.

Additional information regarding DARPins and other DRP technologies can be found in U.S. Patent Application Publication No. 2004/0132028 and International Patent Application Publication No. WO 02/20565, both of which are hereby incorporated by reference in their entirety.

Anticalins are an additional antibody mimetic technology, however in this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids which is marginally larger than a single immunoglobulin domain.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein can be isolated and binding affinities in the nanomolar or higher range can be obtained.

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains.

Modulation of multiple targets through a single molecule is particularly advantageous in diseases known to involve more than a single causative factor. Moreover, bi- or multivalent binding formats such as Duocalins have significant potential in targeting cell surface molecules in disease, mediating agonistic effects on signal transduction pathways or inducing enhanced internalization effects via binding and clustering of cell surface receptors. Furthermore, the high intrinsic stability of Duocalins is comparable to monomeric Anticalins, offering flexible formulation and delivery potential for Duocalins.

Additional information regarding Anticalins can be found in U.S. Pat. No. 7,250,297 and International Patent Application Publication No. WO 99/16873, both of which are hereby incorporated by reference in their entirety.

Another antibody mimetic technology useful in the context of the instant invention are Avitners. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets.

Additional information regarding Avimers can be found in U.S. Patent Application Publication Nos. 2006/0286603, 2006/0234299, 2006.0223114, 2006/01778.31, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller; more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

The inspiration for Versabodies comes from the natural injectable biopharmaceuticals produced by leeches, snakes, spiders, scorpions, snails, and anemones, which are known to exhibit unexpectedly low immunogenicity. Starting with selected natural protein families, by design and by screening the size, hydrophobicity, proteolytic antigen processing, and epitope density are minimized to levels far below the average for natural injectable proteins.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life.

Additional information regarding Versabodies can be found in U.S. Patent Application Publication No. 2007/0191272 which is hereby incorporated by reference in its entirety.

SMIPs™ (Small Modular ImmunoPharmnaceuticals-Trubion Pharmaceuticals) engineered to maintain and optimize target binding, effector functions, in vivo half life, and expression levels. SMIPS consist of three distinct modular domains. First they contain a binding domain which may consist of any protein which confers specificity (e.g., cell surface receptors, single chain antibodies, soluble proteins, etc). Secondly, they contain a hinge domain which serves as a flexible linker between the binding domain and the effector domain, and also helps control multimerization of the SMIP drug. Finally, SMIPS contain an effector domain which may be derived from a variety of molecules including Fc domains or other specially designed proteins. The modularity of the design, which allows the simple construction of SMIPs with a variety of different binding, hinge, and effector domains, provides for rapid and customizable drug design.

More information on SMIPs, including examples of how to design them, may be found in Zhao et al. (2007) Blood 110:2569-77 and the following U.S. Pat. App. Nos. 20050238646; 20050202534; 20050202028; 200502023; 20050202012; 20050186216; 20050180970; and 20050175614.

The detailed description of antibody fragment and antibody mimetic technologies provided above is not intended to be a comprehensive list of all technologies that could be used in the context of the instant specification. For example, and also not by way of limitation, a variety of additional technologies including alternative polypeptide-based technologies, such as fusions of complimentary determining regions as outlined in Qui et al., Nature Biotechnology, 25(8) 921-929 (2007), which is hereby incorporated by reference in its entirety, as well as nucleic acid-based technologies, such as the RNA aptamer technologies described in U.S. Pat. Nos. 5,789,157, 5,864,026, 5,712,375, 5,763,566, 6,013,443, 6,376,474, 6,613,526, 6,114,120, 6,261,774, and 6,387,620, all of which are hereby incorporated by reference, could be used in the context of the instant invention.

B. Immunoconjugates

In another aspect, the methods of present invention employ immunoconjugate agents that target BMP9 or BMP10 and which inhibit or down-modulate BMP9 or BMP10. Agents that can be targeted to BMP9 or BMP10 include, but are not limited to, cytotoxic agents, anti-inflammatory agents, e.g., a steroidal or nonsteroidal inflammatory agent, or a cytotoxin antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomanitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The term “cytotoxin” or “cytotoxic agent” includes any agent that is detrimental (e.g., kills) to fibrotic tissue. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Immunoconjugates can be formed by conjugating (e.g., chemically linking or recombinantly expressing) antibodies to suitable therapeutic agents. Suitable agents include, for example, a cytotoxic agent, a toxin (e.g. an enzymatically active toxin of bacterial, fungal, plant or animal origin, or fragments thereof), and/or a radioactive isotope (i.e., a radioconjugated). Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, nmitogellin, restrictocin, phenomycin, enomycin and the tricothecenes. A variety of radionuclides are available for the production of radioconjugated antibodies. Examples include ²¹²Bi, ¹³¹I, ¹³¹In, ⁹⁰Y and ¹⁸⁶Re.

Immunoconjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinirnidyl suberate), aldehydes (such as glutareldehyde), his-azido compounds (such as his (p-azidobenzoyl) hexanediamine), bis-diazonium derivatives (such as bis-(p-diazoniumbenzoyl)-ethylenediamine), diisocyanates (such as tolyene 2,6-diisocyanate), and his-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody (see, e.g., WO94/11026).

C. Small Molecule Inhibitors

In another embodiment, the BMP9 or BMP10 antagonists employed in the methods of the invention are small molecules. As used herein, the term “small molecule” is a term of the art and includes molecules that are less than about 7500, less than about 5000, less than about 1000 molecular weight or less than about 500 molecular weight, and inhibit BMP9 or BMP10 activity. Exemplary small molecules include, but are not limited to, small organic molecules (e.g., Cane et al. 1998. Science 282:63), and natural product extract libraries. In another embodiment, the compounds are small, organic non-peptidic compounds. Like antibodies, these small molecule inhibitors indirectly or directly inhibit the activity of BMP9 or BMP10.

D. Nucleic Acids/Antisense Molecules

In another embodiment, the BMP9 or BMP10 antagonist employed in the methods of the present invention is an antisense nucleic acid molecule that is complementary to a gene encoding BMP9 or BMP10, or to a portion of that gene, or a recombinant expression vector encoding the antisense nucleic acid molecule. As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

The use of antisense nucleic acids to down-modulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J. J. (1995) Br. Med. Bull. 51:217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Antisense nucleic acids can be designed according to the rules of Watson and Crick base pairing. The antisense nucleic acid molecule can be complementary to the entire coding region of BMP9 or BMP10 mRNA, but more preferably is an oligonucleotide which is antisense to only a portion of the coding or noncoding region of BMP9 or BMP10 mRNA. For example, the antisense oligonucleotide can be complementary to the region surrounding the translation start site of BMP9 or BMP10 mRNA. An antisense oligonucleotide can be, for example, about 5, 10, 15, 20, 25, 30, 35, 40, 45 or 50 nucleotides in length. An anrisense nucleic acid can be constructed using chemical synthesis and enzymatic ligation reactions using procedures known in the art. For example, an antisense nucleic acid (e.g. an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyl adenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules that can be utilized in the methods of the present invention are typically administered to a subject or generated in situ such that they hybridize with or bind to cellular mRNA and/or genomic DNA encoding BMP9 or BMP10 to thereby inhibit expression by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using vectors well known in the art and described in, for example, US20070111230 the entire contents of which are incorporated herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule employed by the methods of the present invention can include an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In another embodiment, an antisense nucleic acid used in the methods of the present invention is a compound that mediates RNAL RNA interfering agents include, but are not limited to, nucleic acid molecules including RNA molecules which are homologous to BMP9 or BMP10 or a fragment thereof, “short interfering RNA” (siRNA), “short hairpin” or “small hairpin RNA” (shRNA), and small molecules which interfere with or inhibit expression of a target gene by RNA interference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g., New England Biolabs and Ambion. In one embodiment one or more of the chemistries described above for use in antisense RNA can be employed.

In still another embodiment, an antisense nucleic acid is a ribozyme. Ribozymes are catalytic RNA molecules with ribonuclease activity which are capable of cleaving a single-stranded nucleic acid, such as an mRNA, to which they have a complementary region. Thus, ribozyvaes (e.g., hammerhead ribozymes (described in Haselhoff and Gerlach, 1.988, Nature 334:585-591) can be used to catalytically cleave BMP9 or BMP10 mRNA transcripts to thereby inhibit translation of BMP9 or BMP10 mRNA.

Alternatively, gene expression can be inhibited by targeting nucleotide sequences complementary to the regulatory region of BMP9 or BMP10 (e.g., the promoter and/or enhancers) to form triple helical structures that prevent transcription of the BMP9 or BMP10 gene. See generally, Helene, C., 1991, Anticancer Drug Des. 6(6):569-84; Helene, C. et al., 1992, Ann. N.Y. Acad. Sci. 660:27-36; and Maher, L. J., 1992, Bioassays 14(12): 807-15.

E. Fusion Proteins and BMP9 or BMP10-Derived Peptidic Compounds

In another embodiment, the BMP9 or BMP10 antagonist used in the methods of the present invention is a fusion protein or peptidic compound derived from the BMP9 or BMP10 amino acid sequence. In particular, the inhibitory compound comprises a fusion protein or a portion of BMP9 or BMP10 (or a mimetic thereof) that mediates interaction of BMP9 or BMP10 with a target molecule (e.g., ALK1) such that contact of BMP9 or BMP10 with this fusion protein or peptidic compound competitively inhibits the interaction of BMP9 or BMP10 with the target molecule. Such fusion proteins and peptidic compounds can be made using standard techniques known in the art. For example, peptidic compounds can be made by chemical synthesis using standard peptide synthesis techniques and then introduced into cells by a variety of means known in the art for introducing peptides into cells (e.g., liposome and the like).

The in vivo half-life of the fusion protein or peptidic compounds of the invention can be improved by making peptide modifications, such as the addition of N-linked glycosylation sites into BMP9 or BMP10, or conjugating BMP9 or BMP10 to poly(ethylene glycol) (PEG; pegylation), e.g., via lysine-monopegylation. Such techniques have proven to be beneficial in prolonging the half-life of therapeutic protein drugs. It is expected that pegylation of the BMP9 or BMP10 polypeptides of the invention may result in similar pharmaceutical advantages.

In addition, pegylation can be achieved in any part of a polypeptide of the invention by the introduction of a nonnatural amino acid. Certain nonnatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. The BMP9 or BMP10 polypeptides containing these novel amino acids can then be pegylated at these chosen sites in the protein.

F. Receptor-Based Antagonists

In another embodiment, the BMP9 or BMP10 antagonist used in the methods of the present invention is a receptor-based antagonist. Receptor-based antagonists include soluble BMP9 and BMP10 receptors that bind BMP9 or BMP10 (or portions thereof), respectively, and disrupt BMP9 and/or BMP10 activity and/or function. In a particular embodiment, the receptor-based antagonist includes, but is not limited to, soluble ALK1 or serine/threonine kinase receptors.

### V. KITS

The invention also provides kits for assessing whether a subject has or is at risk for developing a fibrotic disorder. These kits include one or more of the following: a detectable moiety, e.g., an antibody or PCR probe, that is able to detect BMP9 and/or BMP10, reagents for obtaining and/or preparing samples for staining, and instructions for use.

The kits of the invention may optionally comprise additional components useful for performing the methods of the invention. By way of example, the kits may comprise fluids (e.g., SSC buffer) suitable for annealing complementary nucleic acids or for binding an antibody with a protein with which it specifically binds, one or more sample compartments, an instructional material which describes performance of a method of the invention and tissue specific controls/standards.

The present invention is further illustrated by the following examples which should not be construed as further limiting.

### EXAMPLES

### Example 1

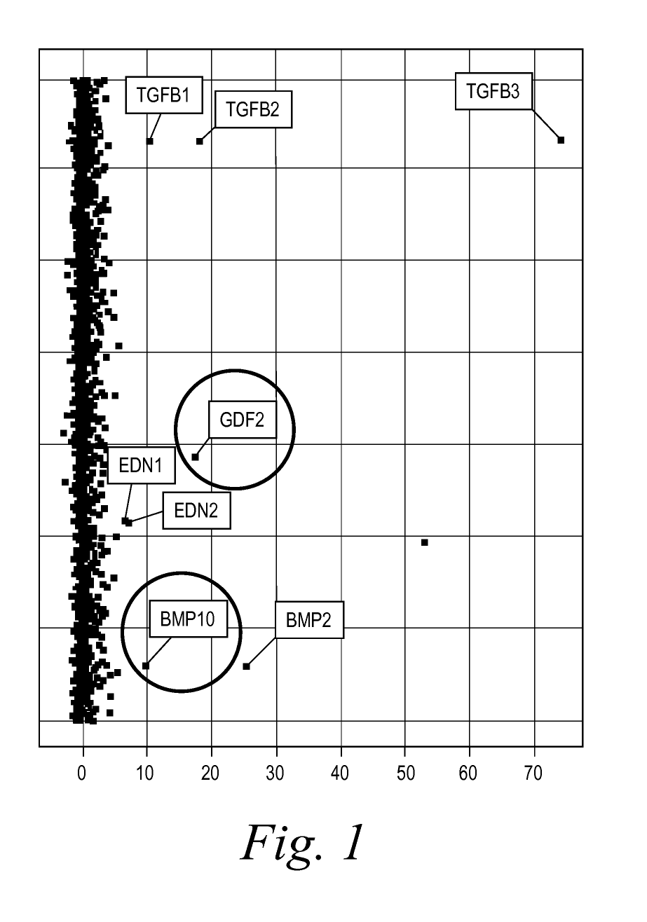

BMP9 and BMP10 were identified as indicators of fibrosis in a phenotypic screen and, therefore, as potential therapeutic targets capable of treating fibrotic disorders. The experiment was performed in 384 well plate format and in triplicate. Briefly, a reverse transfection in Hek293 cells of approximately 1500 human cDNA expression clones encoding predicted secreted proteins was performed using Fugene 6 (Roche) at a ratio of 4:1 (Fugene:DNA) in complete DMEM (Invitrogen) medium. Hek293 cells were washed 24 hours post transfection and incubated for an additional 48 hours to enrich for secreted proteins. Conditioned media from the transfected Hek293 cells were transferred onto human primary dermal fibroblast at passage 3 (Lonza) that were synchronized by starvation for 24 hours in absence of serum. The differentiation of fibroblast into myofibroblast was scored 96 hours later by immunostaining for the presence of alpha smooth muscle actin (αSMA [Sigma], a known marker of fibroblast differentiation) using high content imaging (FIG. 1). Three fields of view were imaged using an in Cell Analyzer (GE Healthcare) and pixel intensity per nuclei was calculated as a measure of percent differentiation.

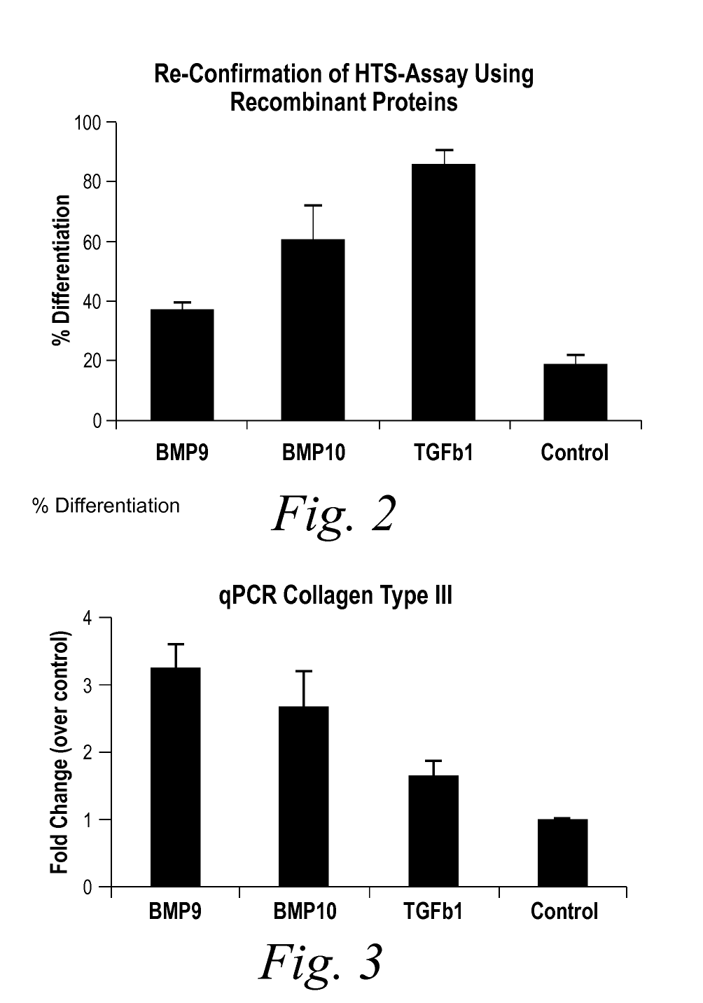

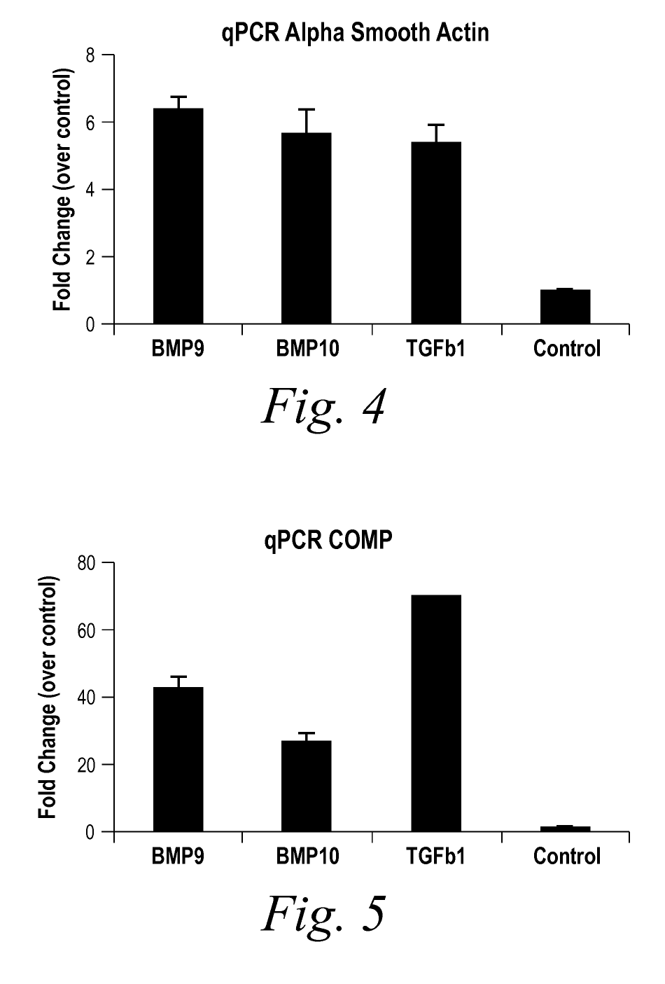

To confirm the activity of BMP9 and BMP10 on fibroblast differentiation, recombinant BMP9 and BMP10 protein was obtained (R&D) and used to stimulate resting fibroblast (FIG. 2). Briefly, 40 ng/ml of active BMP9 recombinant protein and 1000 ng/ml of active BMP10 recombinant protein (R&D System) were serially diluted 2 fold and used in a 12 dose response experiment using human primary dennal fibroblast at passage 3 that were synchronized by starvation for 24 hours in absence of serum. The differentiation of fibroblast into myofibroblast was scored 96 hours later by immunostaining for the presence of alpha smooth muscle actin using high content imaging. BMP9 and BMP10 were shown to positively modulate differentiation of fibroblasts at an effective concentration (EC50) of approximately 2 ng/ml and approximately 125 ng/ml, respectively. In addition, treatment of fibroblasts with recombinant BMP9 or BMP10 proteins used at an EC90 dose of 10 ng/ml and 250 ng/ml respectively for 24-96 hours also led to the up-regulation of known markers of fibrosis. Briefly, human primary dermal fibroblast at passage 3 that were synchronized by starvation for 24 hours in absence of serum then treated with BMP9, BMP10 or with 10 ng/ml TGFb 1. Fibroblasts were collected at various time post stimulation (24-96 hours) and RNAs were prepared (Qiagen) for use in qPCR assays. Modulation of known fibrotic markers including alpha smooth muscle actin, collagen type III and cartilage oligomeric matrix protein (COMP) by TGFb, BMP9 or BMP10 was determined (FIGS. 3-5). Both BMP9 and BMP10 were shown to induce transcriptional activity of alpha smooth muscle actin, collagen type III and cartilage oligomeric matrix protein.

### Example 2

To further assess the role of BMP9 in fibrosis, a reporter gene assay (RGA) was established. Specifically, a BMP9 reporter construct (ID-BRE) was generated using Id1 promoter BMP-response element (BRE) fused with firefly luciferase gene. The BMP9 RGA was conducted on HEK293 cells stably transfected with ID-BRE. HEK293 ID-BRE cells were seeded at 5×10⁵ cells/35 mm-dish one day before transfection. 2.5 μg DNA constructs of constitutively active ALK1 and GFP (10:1) were co-transfected into HEK293 ID-BRE cells via Fugene 6 (Roche, 8 μl), followed by manufacturer's protocol on day 2. Transfection efficiency was measured by visualization of GFP. Transfected cells were then seeded into 96-well plate at 1×10⁴ cells/well on day 3. BMP9 was added to the 96-well plate at different concentration with or without a BMP9 neutralizing antibody or a soluble receptor, ALK1-Fc, on day 4. Cells were harvested on day 5 and luciferase activity was measured using Bright-Glo Luciferase Assay System (Promega) following manufacturer's protocol.

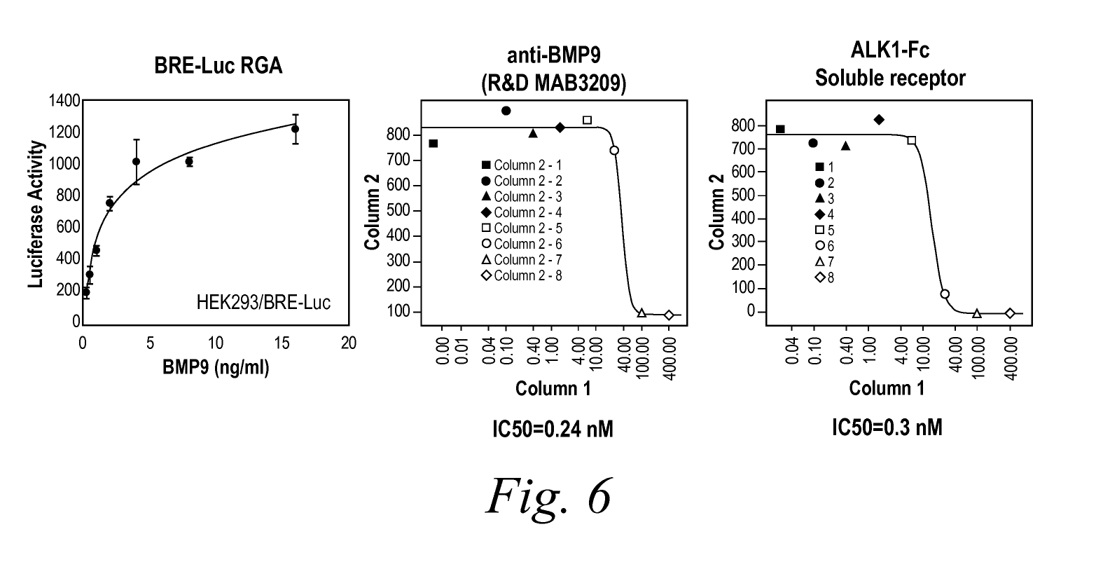

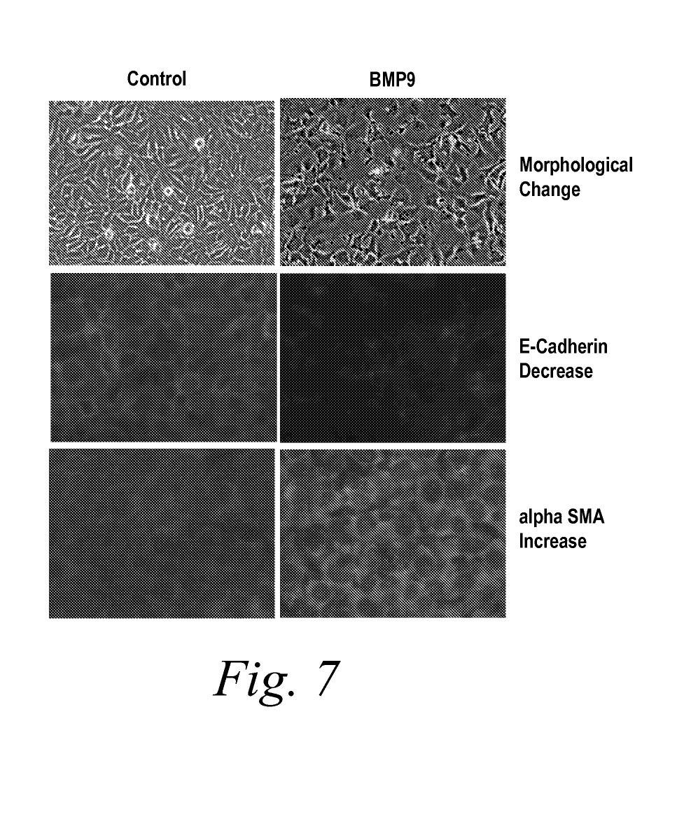

As shown in FIG. 6, both an anti-BMP9 neutralizing antibody and a soluble receptor, ALK1-Fc, inhibited BMP9-stimulated BRE-luciferase activity.

### Example 3

An Epithelial-Mesenchymal Transdiferentiation (EMT) assay was performed to assess whether BMP9 has any effect on EMT in hepatocytes. Specifically, Hep3B cells (ATCC) were cultured in DMEM-F12, supplemented with 10% FBS, 10 U/ml penicillin, and 10 U/ml streptomycin, at 37° C. in a humidified atmosphere containing 5% CO2. 2×10⁵ Hep3B cells were seeded in complete DMEM-F12 into each well of 24-well plates.

The medium was replaced with low serum (1%) medium with or without 10 ng/ml of BMP9 (R&D).

In one experiment, the cells were fixed 24 hours post-treatment with or without BMP9, fixed for image analysis and subjected to immunohistochemistry staining with E-cadherin and α-SMA. As evidenced by the cell morphological changes depicted in FIG. 6, BMP9 induced EMT, decreased E-cadherin and increased α-SMA, as compared to the control.

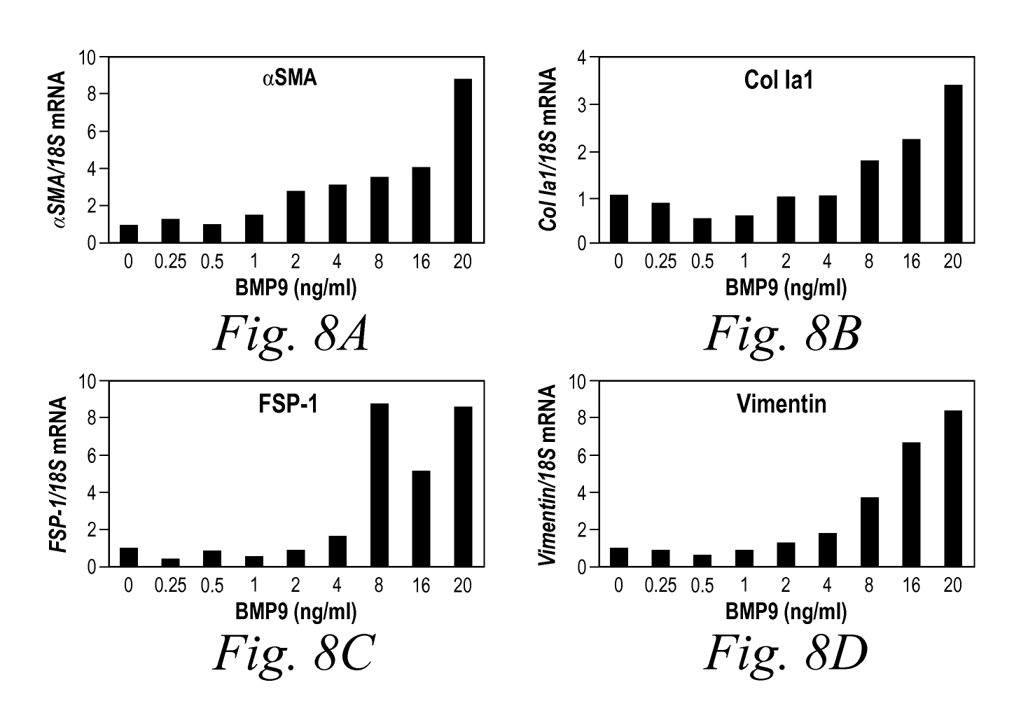

For quantitative PCR analysis, the cells were harvested 48 hours post-BMP9 treatment and the total RNA was extracted and subjected to RT-PCR analysis. The PCR was run in ABI7700HT. As shown in FIGS. 8A-8D, BMP9 induced expression of four different fibroblast markers (i.e., αSMA, Col la1, fibroblast specific protein (FSP-1), and vimentin) in a dose-dependent manner.

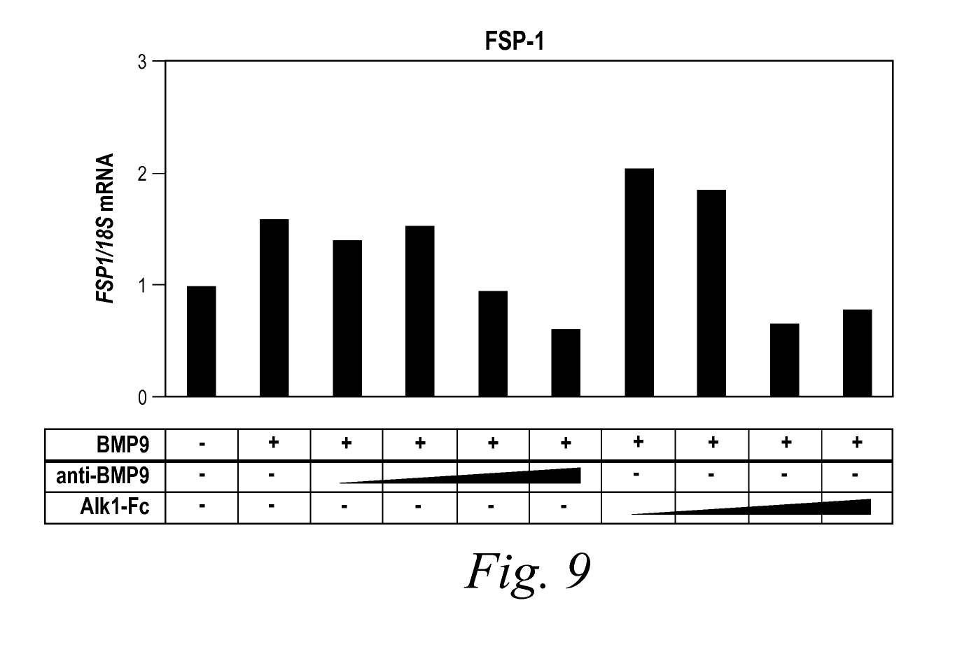

The cells were also treated with BMP9 in the absence or presence of an anti-BMP9 antibody or ALK1-Fc. 48 hours post-treatment, the total fibroblast specific protein (FSP-1) RNA was extracted and subjected for qPCR analysis. As shown in FIG. 9, both a BMP9 neutralizing monoclonal antibody and a soluble receptor (ALK1-Fc) inhibited BMP9-induced FSP-1 expression.

### EQUIVALENTS

Those skilled in the art will recognize, or be able to ascertain using no more than routine experimentation, many equivalents of the specific embodiments of the invention described herein. Such equivalents are intended to be encompassed by the following claims. Any combination of the embodiments disclosed in the dependent claims are contemplated to be within the scope of the invention.

### INCORPORATION BY REFERENCE

The contents of all references, patents and published patent applications cited throughout this application, as well as the figures and Sequence Listing, are expressly incorporated herein by reference.

1. A method of inhibiting fibrosis in a cell, comprising contacting said cell with an effective amount of a BMP9 or BMP10 antagonist, thereby inhibiting fibrosis in said cell.

2. The method of claim 1, wherein said cell is selected from the group consisting of a pulmonary cell, a liver cell, a kidney cell, a cardiac cell, a musculoskeletal cell, a skin cell, an eye cell, and a pancreatic cell.

3. A method of treating a fibrotic disorder in a subject, comprising administering to said subject an effective amount of a BMP9 or BMP10 antagonist, thereby treating a fibrotic disorder in said subject.

4. A method of preventing a fibrotic disorder in a subject, comprising administering to said subject an effective amount of a BMP9 or BMP10 antagonist, thereby preventing a fibrotic disorder in said subject.

5. The method of claim 3, wherein the fibrotic disorder is selected from the group consisting of vascular fibrosis, pulmonary fibrosis, pancreatic fibrosis, liver fibrosis, renal fibrosis, musculoskeletal fibrosis, cardiac fibrosis, skin fibrosis, eye fibrosis, glaucoma, progressive systemic sclerosis (PSS), chronic graft versus-host disease, scleroderma, Peyronie's disease, post-cystoscopic urethral stenosis, idiopathic and pharmacologically induced retroperitoneal fibrosis, mediastinal fibrosis, progressive massive fibrosis, proliferative fibrosis and neoplastic fibrosis.

6. The method of claim 3 further comprising administering to said subject an additional therapeutic agent.

7. The method of claim 3, wherein said subject is human.

8. (canceled)

9. The method of claim 5, wherein the BMP9 or BMP10 antagonist is selected from the group consisting of an antibody, a small molecule, a nucleic acid, a fusion protein, an adnectin, an aptamer, an anticalin, a lipocalin, a BMP9 or BMP10-derived peptidic compound, and a receptor-based antagonist.

10.-15. (canceled)

16. A method of inhibiting the differentiation of a fibroblast to a myofibroblast, comprising contacting a fibroblast with an effective amount of a BMP9 or BMP10 antagonist, thereby inhibiting the differentiation of a fibroblast to a myofibroblast.

17. The method of claim 16, wherein the BMP9 or BMP10 antagonist is selected from the group consisting of an antibody, a small molecule, a nucleic acid, a fusion protein, an adnectin, an aptamer, an anticalin, a lipocalin, and a BMP9 or BMP10-derived peptidic compound.

18.-20. (canceled)

21. A method for assessing whether a subject has or is at risk of developing a fibrotic disorder comprising: (i) contacting a sample from said subject with a reagent able to detect BMP9 or BMP10; and (ii) detecting BMP9 or BMP10, wherein an elevated level of BMP9 or BMP10 relative to a control is an indication that the subject has or is at risk of developing a fibrotic disorder.

22.-30. (canceled)

31. A method of assessing the efficacy of a treatment regimen for treating a fibrotic disorder in a subject, the method comprising:
a) contacting a first sample obtained from said subject prior to administering at least a portion of the treatment regimen to the subject with a reagent able to detect BMP9 or BMP10;
b) contacting a second sample obtained from said subject following administration of at least a portion of the treatment regimen with a reagent able to detect BMP9 or BMP10; and
c) comparing the levels of BMP9 or BMP10 from the first and second samples, wherein an elevated level of BMP9 or BMP10 present in the first sample, relative to the second sample, is an indication that the treatment regimen is efficacious for treating a fibrotic disorder in the subject.

32. The method of claim 31, wherein the treatment regimen comprises administration of an BMP9 or BMP10 antagonist.

33.-37. (canceled)

38. A method of inhibiting or preventing epithelial-mesenchymal transition (EMT) in a subject comprising administering to the subject an effective amount of BMP9 or BMP10 antagonist, thereby inhibiting or preventing epithelial-mesenchymal transition (EMT).

39. The method of claim 38, wherein the EMT is associated with fibrosis.

40. The method of claim 39, wherein the fibrosis is selected from the group consisting of vascular fibrosis, pulmonary fibrosis, pancreatic fibrosis, liver fibrosis, renal fibrosis, musculoskeletal fibrosis, cardiac fibrosis, skin fibrosis, eye fibrosis, glaucoma, progressive systemic sclerosis (PSS), chronic graft versus-host disease, scleroderma, Peyronie's disease, post-cystoscopic urethral stenosis, idiopathic and pharmacologically induced retroperitoneal fibrosis, mediastinal fibrosis, progressive massive fibrosis, proliferative fibrosis and neoplastic fibrosis.

41. The method of claim 38, wherein the BMP9 or BMP10 antagonist is selected from the group consisting of an antibody, a small molecule, a nucleic acid, a fusion protein, an adnectin, an aptamer, an anticalin, a lipocalin, a BMP9 or BMP10-derived peptidic compound, and a receptor-based antagonist.

42.-47. (canceled)

48. A method of identifying human liver cell sample as being fibrosis comprising detecting BMP9 expression level in said cell sample with a reagent, wherein an elevated level of BMP9 relative to normal human liver cells is an indication that said liver cell sample has fibrosis.

49. The method of claim 48 further comprising detecting an additional fibrosis marker selected from the group consisting of alpha smooth muscle actin, collagen type III cartilage oligomeric matrix protein, collagen type I, collagen type IV, fibroblast specific protein-1, fibronectin, serpinE1, periostin, IGFBP3, SPARC, CTGF, TGFb, Cyr61, and phospho smad 2/3.

50. The method of claim 48, wherein the reagent is an antibody or a nucleic acid.

51. The method of claim 48, wherein the reagent is detectably labeled.

52. The method of claim 51, wherein the label is selected from the group consisting of a radioisotope, a bioluminescent compound, a chemiluminescent compound, a fluorescent compound, a metal chelate, or an enzyme.

53. The method of claim 48, wherein the level of BMP9 is at least 2-fold higher relative to normal human liver cells.

54. The method of claim 48, wherein the level of BMP9 is at least 3-fold higher relative to normal human liver cells.


SEQUENCE LISTING (4 sequences)

SEQ ID NO: 1 - protein, 429 aa, Homo sapiens
MCPGALWVALPLLSLLAGSLQGKPLQSWGRGSAGGNAHSPLGVPGGGLPEHTFNLKMFLENVKVDFLRSLNLSGVPSQDKTRVEPPQYMIDLYNRYTSDKSTTPASNIVRSFSMEDAISITATEDFPFQKHILLFNISIPRHEQITRAELRLYVSCQNHVDPSHDLKGSVVIYDVLDGTDAWDSATETKTFLVSQDIQDEGWETLEVSSAVKRWVRSDSTKSKNKLEVTVESHRKGCDTLDISVPPGSRNLPFFVVFSNDHSSGTKETRLELREMISHEQESVLKKLSKDGSTEAGESSHEEDTDGHVAAGSTLARRKRSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 2 - DNA, 1942 bp, Homo sapiens
cggtccagcccggcagcgggtgagagtgggtgctggccaggacggttccttcagagcaaacagcagggagatgccggcccgctccttcccagctcctccccgtgcccgctaacacagcacggccgcctgcagtctcctctctgggtgattgcgcgggcctaagatgtgtcctggggcactgtgggtggccctgcccctgctgtccctgctggctggctccctacaggggaagccactgcagagctggggacgagggtctgctgggggaaacgcccacagcccactgggggtgcctggaggtgggctgcctgagcacaccttcaacctgaagatgtttctggagaacgtgaaggtggatttcctgcgcagccttaacctgagtggggtcccttcgcaggacaaaaccagggtggagccgccgcagtacatgattgacctgtacaacaggtacacgtccgataagtcgactacgccagcgtccaacattgtgcggagcttcagcatggaagatgccatctccataactgccacagaggacttccccttccagaagcacatcttgctcttcaacatctccattcctaggcatgagcagatcaccagagctgagctccgactctatgtctcctgtcaaaatcacgtggacccctctcatgacctgaaaggaagcgtggtcatttatgatgttctggatggaacagatgcctgggatagtgctacagagaccaagaccttcctggtgtcccaggacattcaggatgagggctgggagaccttggaagtgtccagcgccgtgaagcgctgggtccggtccgactccaccaagagcaaaaataagctggaagtgactgtggagagccacaggaagggctgcgacacgctggacatcagtgtccccccaggttccagaaacctgcccttctttgttgtcttctccaatgaccacagcagtgggaccaaggagaccaggctggagctgagggagatgatcagccatgaacaagagagcgtgctcaagaagctgtccaaggacggctccacagaggcaggtgagagcagtcacgaggaggacacggatggccacgtggctgcggggtcgactttagccaggcggaaaaggagcgccggggctggcagccactgtcaaaagacctccctgcgggtaaacttcgaggacatcggctgggacagctggatcattgcacccaaggagtatgaagcctacgagtgtaagggcggctgcttcttccccttggctgacgatgtgacgccgacgaaacacgctatcgtgcagaccctggtgcatctcaagttccccacaaaggtgggcaaggcctgctgtgtgcccaccaaactgagccccatctccgtcctctacaaggatgacatgggggtgcccaccctcaagtaccattacgagggcatgagcgtggcagagtgtgggtgcaggtagtatctgcctgcggggctggggaggcaggccaaaggggctccacatgagaggtcctgcatgcccctgggcacaacaaggactgattcaatctgcatgccagcctggaggaggaaagggagcctgctctccctccccacaccccacccaaagcatacaccgctgagctcaactgccagggaaggctaaggaaatggggatttgagcacaacaggaaagcctgggagggttgttgggatgcaaggaggtgatgaaaaggagacagggggaaaaataatccatagtcagcagaaaacaacagcagtgagccagaggagcacaggcgggcaggtcactgcagagactgatggaagttagagaggtggaggaggccagctcgctccaaaacccttggggagtagagggaaggagcaggccgcgtgtcacacccatcattgtatgttatttcccacaacccagttggaggggcatggcttccaatttagagacccg

SEQ ID NO: 3 - protein, 414 aa, Homo sapiens
MGSLVLTLCALFCLAAYLVSGSPIMNLEQSPLEEDMSLFGDVFSEQDGVDFNTLLQSMKDEFLKTLNLSDIPTQDSAKVDPPEYMLELYNKFATDRTSMPSANIIRSFKNEDLFSQPVSFNVSIPHHEEVIMAELRLYTLVQRDRMIYDGVDRKITIFEVLESKGDNEGERNMLVLVSGEIYGTNSEWETFDVTDAIRRWQKSGSSTHQLEVHIESKHDEAEDASSGRLEIDTSAQNKHNPLLIVFSDDQSSDKERKEELNEMISHEQLPELDNLGLDSFSSGPGEEALLQMRSNIIYDSTARIRRNAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 4 - DNA, 1584 bp, Homo sapiens
ggggagaggaagagtggtagggggagggagagagagaggaagagtttccaaacttgtctccagtgacaggagacatttacgttccacaagataaaactgccacttagagcccagggaagctaaaccttcctggcttggcctaggagctcgagcggagtcatgggctctctggtcctgacactgtgcgctcttttctgcctggcagcttacttggtttctggcagccccatcatgaacctagagcagtctcctctggaagaagatatgtccctctttggtgatgttttctcagagcaagacggtgtcgactttaacacactgctccagagcatgaaggatgagtttcttaagacactaaacctctctgacatccccacgcaggattcagccaaggtggacccaccagagtacatgttggaactctacaacaaatttgcaacagatcggacctccatgccctctgccaacatcattaggagtttcaagaatgaagatctgttttcccagccggtcagttttaatgggctccgaaaataccccctcctcttcaatgtgtccattcctcaccatgaagaggtcatcatggctgaacttaggctatacacactggtgcaaagggatcgtatgatatacgatggagtagaccggaaaattaccatttttgaagtgctggagagcaaaggggataatgagggagaaagaaacatgctggtcttggtgtctggggagatatatggaaccaacagtgagtgggagacttttgatgtcacagatgccatcagacgttggcaaaagtcaggctcatccacccaccagctggaggtccacattgagagcaaacacgatgaagctgaggatgccagcagtggacggctagaaatagataccagtgcccagaataagcataaccctttgctcatcgtgttttctgatgaccaaagcagtgacaaggagaggaaggaggaactgaatgaaatgatttcccatgagcaacttccagagctggacaacttgggcctggatagcttttccagtggacctggggaagaggctttgttgcagatgagatcaaacatcatctatgactccactgcccgaatcagaaggaacgccaaaggaaactactgtaagaggaccccgctctacatcgacttcaaggagattgggtgggactcctggatcatcgctccgcctggatacgaagcctatgaatgccgtggtgtttgtaactaccccctggcagagcatctcacacccacaaagcatgcaattatccaggccttggtccacctcaagaattcccagaaagcttccaaagcctgctgtgtgcccacaaagctagagcccatctccatcctctatttagacaaaggcgtcgtcacctacaagtttaaatacgaaggcatggccgtctccgaatgtggctgtagatagaagaagagtcctatggcttatttaataactgtaaatgtgtatatttggtgttcctatttaatgagattatttaataagggtgtacagtaatagaggcttgctgccttcaggaaatggacaggtcagtttgttgtaggaaatgcatatttt